(19) United States
(12) Patent Application Publication
(10) Pub. No.: US 2025/0058015 A1
(43) Pub. Date: Feb. 20, 2025

(54) STARCH-BASED SHAPE MEMORY POLYMER HYDROGELS

(71) Applicants: Mary Beth Monroe, Syracuse, NY (US); Henry T. Beaman, Somerville, NY (US)

(72) Inventors: Mary Beth Monroe, Syracuse, NY (US); Henry T. Beaman, Somerville, NY (US)

(73) Assignee: Syracuse University, Syracuse, NY (US)

(21) Appl. No.: 18/720,725

(22) PCT Filed: Dec. 19, 2022
(86) PCT No.: PCT/US22/53346

§ 371 Date: Jun. 17, 2024

Related U.S. Application Data
(60) Provisional application No. 63/290,713, filed on Dec. 17, 2021.

(51) Int. Cl. A61L 24/04; A61L 24/00

(52) U.S. Cl. CPC A61L 24/043; A61L 24/0031; A61L 24/0036; A61L 24/0042; A61L 2400/16; A61L 2430/36

(57) ABSTRACT

A shape memory polymer scaffold for treatment of a fistula. The scaffold is formed from a shape memory polymer foam that can be triggered to expand from a compressed shape to an expanded shape in response to hydration and/or heating. The shape memory polymer foam includes an antibacterial component and a biodegradable component so that the scaffold addresses infective agents in the fistula and will degrade over time as the fistula heals. The foam may comprise poly(vinyl alcohol), starch, and a phenolic acid.

### CROSS-REFERENCE TO RELATED APPLICATIONS

The present application claims priority to U.S. Provisional Application No. 63/290,713, filed on Dec. 17, 2021.

### BACKGROUND OF THE INVENTION

### 1. Field of the Invention

The present invention relates to shape memory polymer systems and, more specifically, to a fistula treatment approach using a shape memory polymer foam.

### 2. Description of the Related Art

Fistulas are abnormal connections between the digestive, urinary, and/or reproductive system that form due to intestinal bowel disease (IBD), childbirth, surgical complications, or cancer. These tunneling sores cause pain, infection, and abscess formation and can ultimately lead to fecal incontinence, anal stenosis, or sepsis. Crohn's disease is the most common type of IBD, affecting >1,000,000 people in the U.S. and Europe, primarily between the ages of 15 and 30. Almost 20% of Crohn's disease patients already have a fistula at the time of their diagnosis, and ˜35% of Crohn's patients develop fistulas at some point. Currently, 83% of Crohn's fistula patients undergo surgery to drain or bypass the fistula openings. Surgery has short-term effectiveness, but ˜23% of these patients ultimately require bowel resections in which a portion of the colon or rectum is surgically removed. An alternate treatment approach is to fill fistulas to seal off the openings and allow healing. Fibrin glue has been employed as a fistula filling material, but it is susceptible to infection and dislodgement due to poor wall integration. Decellularized porcine scaffolds have also been used as fistula plugs but have a ˜50% failure rate, in part due to infectious abscess formation.

In addition to complications from infection from bacteria in the intestinal tract, an observed epithelial to mesenchymal transition (EMT) prevents fistula healing. The EMT occurs when epithelial cells that line the intestinal tract lose their polarity and acquire migratory function. These migratory epithelial cells move from the intestinal basement membrane into the linings of fistulas. There, they further convert into proliferative mesenchymal-like cells that overproduce fibroblasts and extracellular matrix. Mesenchymal stem cells (MSCs) delivered to the fistula tract have shown some success in closing fistulas, which may be due to their ability to reverse the EMT. However, a lack of proper scaffolding for these cells likely reduces their retention in the fistula site and overall efficacy. Current fistula filling treatments fail to heal fistulas because they do not address infection and the EMT, and MSC treatments that may reverse the EMT are limited by poor delivery vehicles. As a result, there is an urgent need for fistula treatments that are antimicrobial to reduce infection while reversing the EMT to allow healing. In the absence of this technology and the corresponding fundamental information gained during its development, the long-term complications of Crohn's disease will continue to rise as its patient population ages.

### BRIEF SUMMARY OF THE INVENTION

The present invention is a synthetic shape memory polymer (SMP) hydrogel foam that includes naturally occurring components, such as antimicrobial and anti-inflammatory phenolic acids (PAs) and enzymatically-degradable starches to reduce infection, reverse the epithelial to mesenchymal transition (EMT), and optionally provide a MSC delivery vehicle for the treatment of fistulas. The shape memory properties of the foam allow for easy application to long fistula tracts, and the enzymatically-degradable starch components degrade in response to healing to eliminate the need for removal of the foam.

In a first embodiment, the present invention is a shape memory polymer scaffold for treatment of a fistula comprised of a shape memory polymer foam that can be triggered to expand from a compressed shape to an expanded shape in response to water and temperature, wherein the shape memory polymer foam comprises an amount of poly(vinyl alcohol), a biodegradable compound, and a phenolic acid and optionally an amount of mesenchymal stem cells entrapped in the shape memory polymer foam. The biodegradable compound may be starch. The phenolic acid may be vanillic acid. The vanillic acid is coupled to the poly(vinyl alcohol) in place of a plurality of pendant hydroxyls of the poly(vinyl alcohol). The poly(vinyl alcohol) with pendant vanillic acid may be crosslinked with the starch to form a hydrogel network. The hydrogel network may have a plurality of pores. The plurality of pores have a pore size of about 200 micrometers. The phenolic acid is selected from the group consisting of cinnamic acid, p-coumaric acid, ferulic acid, sinapic acid, caffeic acid, benzoic acid, 4-hydroxy benzoic acid, vanillic acid, syringic acid, protocatechuic acid, and gallic acid.

In another aspect, the present invention may be a method of treating a fistula, that begins with the step of providing a shape memory polymer foam and amount of mesenchymal stem cells entrapped in the shape memory polymer foam in a compressed shape, wherein the shape memory polymer foam comprises an amount of poly(vinyl alcohol), a degradable compound, and a phenolic acid. The shape memory polymer foam may then be positioned into a fistula. The shape memory polymer foam is then allowed to expand into an expanded shape from the compressed shape while positioned in the fistula.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic of a scaffold according to the present invention for treating a fistula.

FIG. 2 is a schematic of the structure of poly(vinyl alcohol) modified with vanillic acid (PVA-VA) and a graph of the colony forming units of E. coli after exposure to hydrogels synthesized with unmodified PVA (control) and PVA with 1 and 2% VA substitution. *p<0.05 vs. control.

FIG. 3 is a series of images of water- and heat-induced shape memory of PVA-starch foams, a chart of PVA-starch foam swelling, compressive modulus, and shape fixity, and a graph of enzymatic degradation in glucoamylase.

FIG. 4 is a schematic of a fabrication approach of a scaffold according to the present invention.

FIG. 5 is a graph of the porosity of various scaffolds according to the present invention.

FIG. 6 is a graph of the pore size of various scaffolds according to the present invention.

FIG. 7 is a series of images of various scaffolds according to the present invention.

FIG. 8 is a graph of the stiffness of various scaffolds when dry and when wet according to the present invention.

FIG. 9 a graph of the shape retention of various scaffolds according to the present invention.

FIG. 10 a graph of the degradability of various scaffolds according to the present invention.

FIG. 11 is a series of graphs of (A) Fourier transform infrared spectra of poly(vinyl alcohol) (PVA) 6 k hydrogels with varied cornstarch content. Swelling profiles of (B) PVA 6 k and (C) PVA 25 k hydrogel films in water at 37° C. Mean±standard deviation displayed (n=3).

FIG. 12 is series of images and graphs of A) Scanning electron micrographs of hydrogel foams. Scale bar applies to all images. (B) Caco-2 cytocompatibility after 24, 72, and 168 h of exposure to hydrogel films. Mean±standard deviation displayed (n=3).

FIG. 13 is a series of graphs of Mechanical properties of hydrogel foams and films. Film (A) elastic modulus and (B) strain at break in the wet state. Compressive modulus of foams in the (C) wet and (D) dry state. Solid bars are PVA 6 k formulations and hatched bars are PVA 25 k formulations. Mean±standard deviation displayed. *p<0.05 relative to corollary PVA 6 k formulation. †p<0.05 relative to all other formulations in the same PVA molecular weight. ‡p<0.05 relative to 1:2 and 1:1 formulations with same PVA molecular weight (n=3 for all). PVA, poly(vinyl alcohol).

FIG. 14 is a series of graphs of thermal and shape memory properties of hydrogel foams. (A) Differential scanning calorimetry traces of PVA 6 k foams. Arrows denote endotherms associated with glass transition (^{⋅}60-85° C.) temperatures (T_{g}). (B) Representative images of volume recovery of 0 CS:1 PVA 6 k foam. (C) Volume recovery profiles of PVA 25 k formulations (n=3). CS, cornstarch; PVA, poly(vinyl alcohol).

FIG. 15 is a series of graphs of the degradation profiles (swollen mass remaining) in (A) PBS, (B) 100 U/ml amylase, (C) 10 mM DTT, and (D) 100 U/ml amylase+10 mM DTT of (left) PVA 6 k and (right) PVA 25 k hydrogel foams (n=3) DTT, dithiothreitol; PBS, phosphate buffered saline; PVA, poly(vinyl alcohol)

FIG. 16 is a series of schematic representation of routes of chemical and physical incorporation of phenolic acids into PVA hydrogels.

FIG. 17 is a pair of graphs of fourier transform infrared spectroscopy (FTIR) of (A) physically incorporated PCA-PVA hydrogels and (B) chemically incorporated PCA-PVA hydrogels.

FIG. 18 is a series of graphs of physically incorporated PA-PVA hydrogel film (A) tensile modulus and (B) elongation at break in the wet state. Chemically incorporated PA-PVA hydrogel film (C) tensile modulus and (D) elongation at break in the wet state. N=3, mean±standard deviation displayed. Red lines indicate control hydrogel (no PAs) values for tensile modulus and elongation at break (79±12 kPa and 1.1±0.5, respectively). Symbols indicate p<0.05 relative to control (*), 1% (‡), and 2% (#) PA hydrogels.

FIG. 19 is a graphs of release rates of physically and chemically incorporated PA-PVA hydrogels with (A) cinnamic acid, (B) p-coumaric acid, and (C) caffeic acid with percent of PA content releases at 28 days provided in legend. N=3, mean±standard deviation displayed

FIG. 20 is a series of graphs of (A) Caco-2 cell viability after 24 hours of incubation with all hydrogel formulations, and (B) cell growth based on relative absorbance (compared with cells at 24 hours) after 7-day incubation with CA and PCA hydrogels. N=3, mean±standard deviation displayed. *p<0.05 relative to control.

FIG. 21 is a series of images of (A) Qualitative observations of E. coli colony forming unit density after incubation with chemically and physically incorporated PA hydrogels. Scale bar of 500 mm applies to all images. (B) Crystal violet biofilm staining in wells surrounding physically and chemically incorporated CA and PCA films. The solid line indicates control PVA hydrogel measurement. *p<0.05 relative to control PVA hydrogel in LB media. (C) SEM images of biofilms on CA, PCA, and control hydrogels. The scale bar applies to all images

FIG. 22 is a graph and series of images of the shape memory properties of CA and PCA hydrogels. (A) Shape fixity as measured by the change in distance between folded sample edges after 24 hours of storage, N=3, mean±standard deviation displayed. (B) Shape recovery (unfolding) after incubation in 37° C. water for 30 minutes.

### DETAILED DESCRIPTION OF THE INVENTION

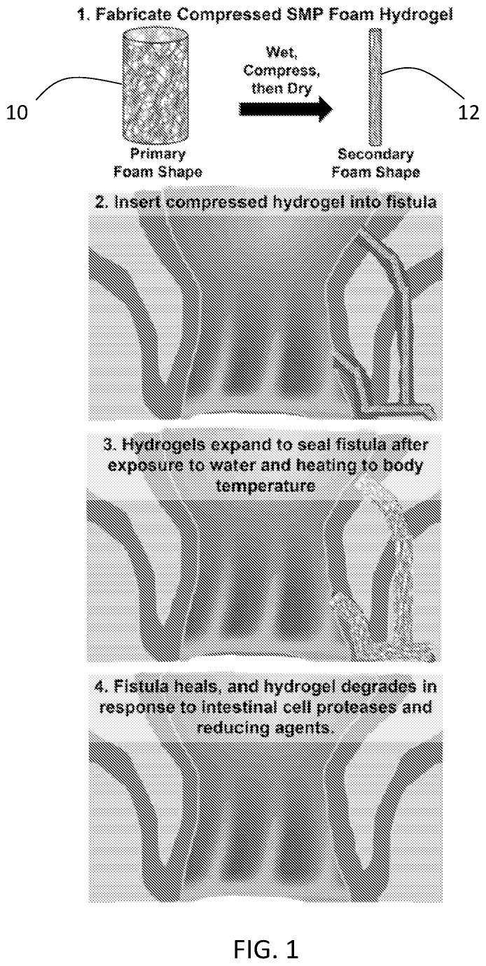

Referring to the figures, wherein like numerals refer to like parts throughout, the present invention comprises a synthetic scaffold 10 comprising a shape memory polymer (SMP) hydrogel foam that includes naturally occurring components, such as antimicrobial and anti-inflammatory phenolic acids (PAs) and enzymatically-degradable starches to reduce infection, reverse the epithelial to mesenchymal transition (EMT), and optionally provide a MSC delivery vehicle for the treatment of fistulas. As seen in FIG. 1, the scaffold may be fabricated and then compressed from an expanded configuration into a compressed configuration. The compressed scaffold may then be inserted into a fistula. The compressed scaffold will expand in response to water and body temperature to fill the fistula. As the fistula heals, the scaffold degrades. The inclusion of antibiotic agents in the scaffold can reduce infections and reverse EMT. A degradable component of the scaffold allows the scaffold to degrade over time as the fistula heals.

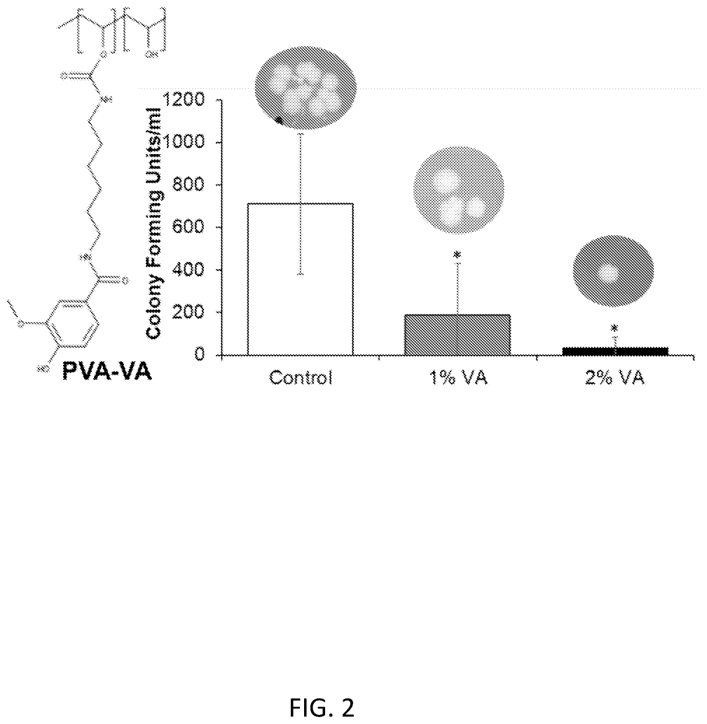

As an example of a foam according to the present invention, poly(vinyl alcohol) (PVA) has been modified with vanillic acid (VA), which has showed high antioxidant activity, cytocompatibility, and antimicrobial properties. PVA is a commonly used hydrogel material due to its cytocompatibility. Its pendant hydroxyls (OH) provide an easy route for modification with phenolic acids, and it exhibits shape memory, which can be employed during fistula delivery. The OH groups were successfully substituted on PVA with pendant VAs to produce PVA-VA, as seen in FIG. 2, which was crosslinked into a PVA/starch hydrogel network. The resulting hydrogels reduced E. coli colony forming units in comparison with control (unmodified PVA) hydrogels. Additional benefits of PAs include antioxidant, pro-coagulant, anti-inflammatory, and estrogenic properties.

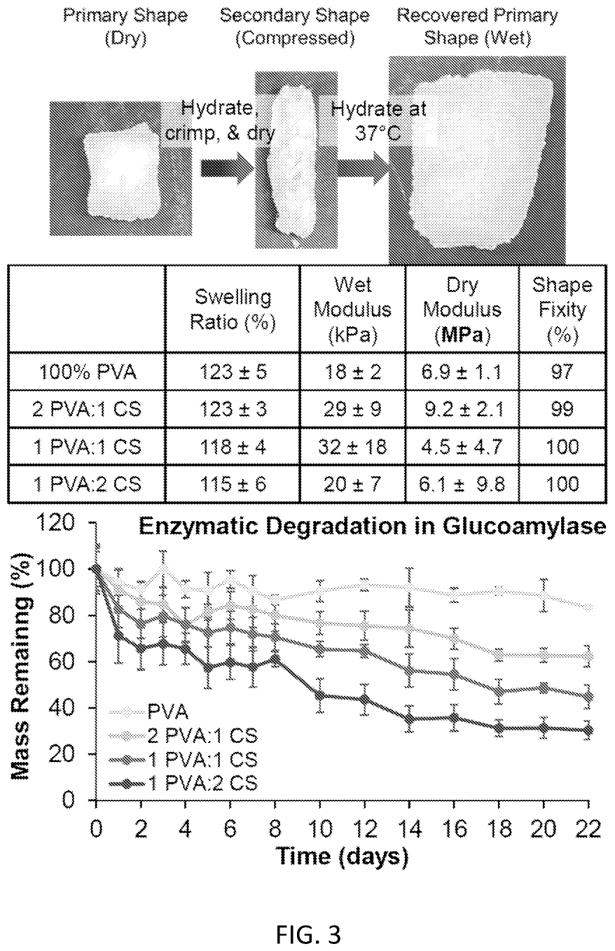

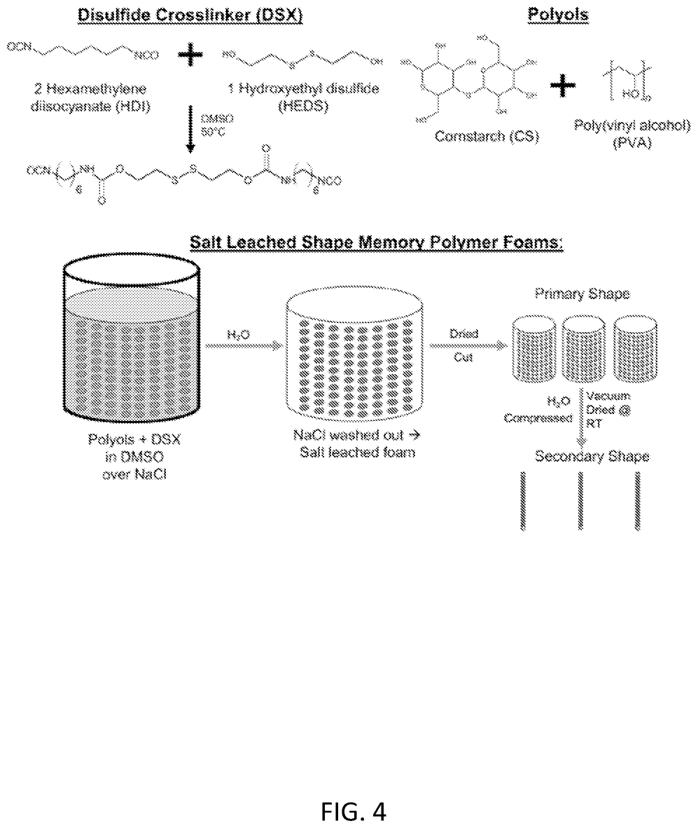

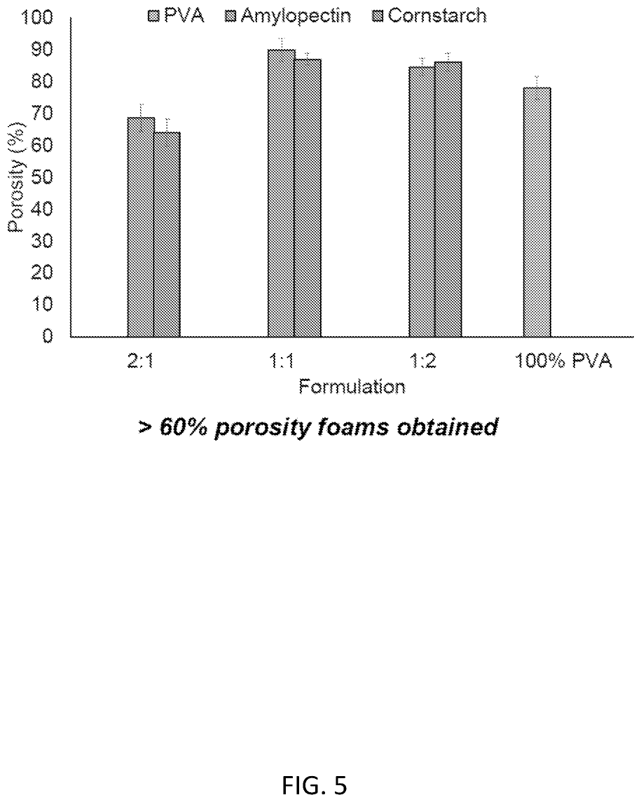

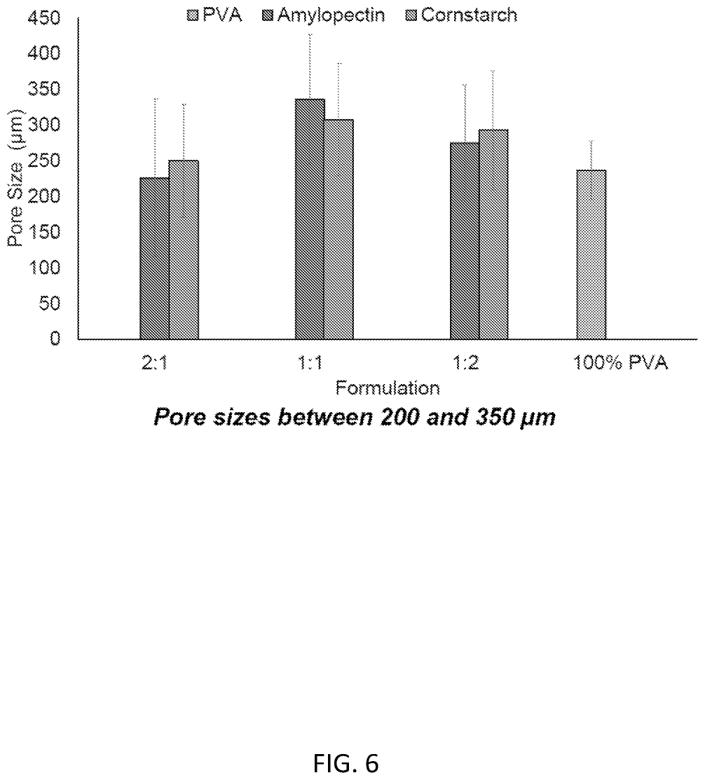

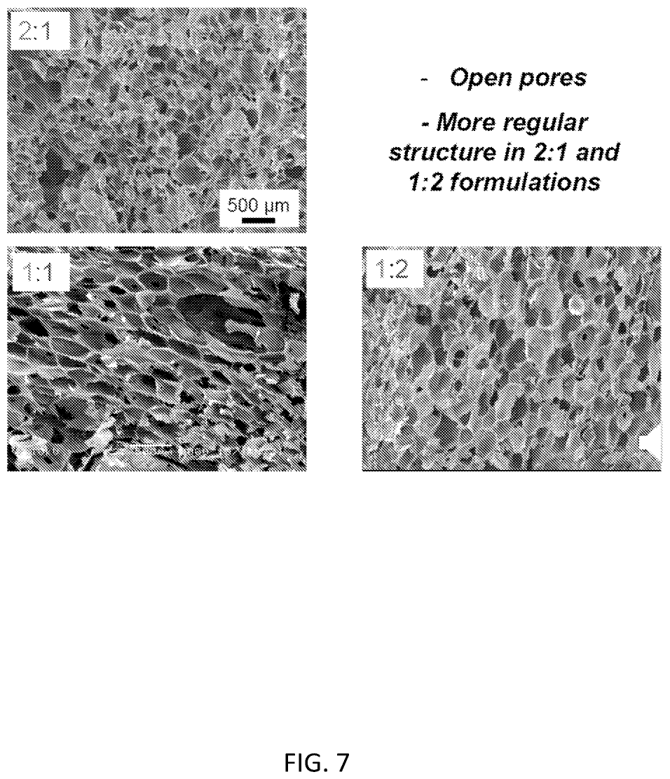

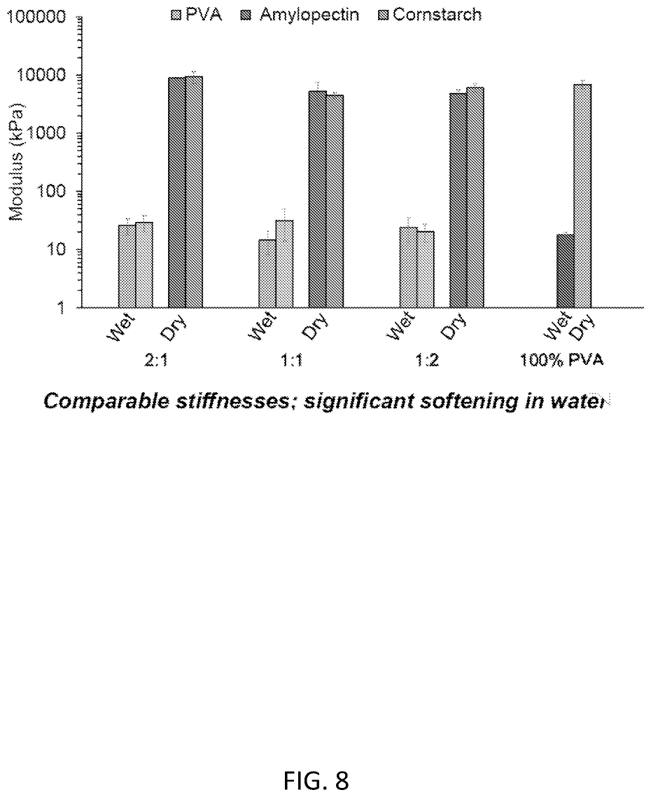

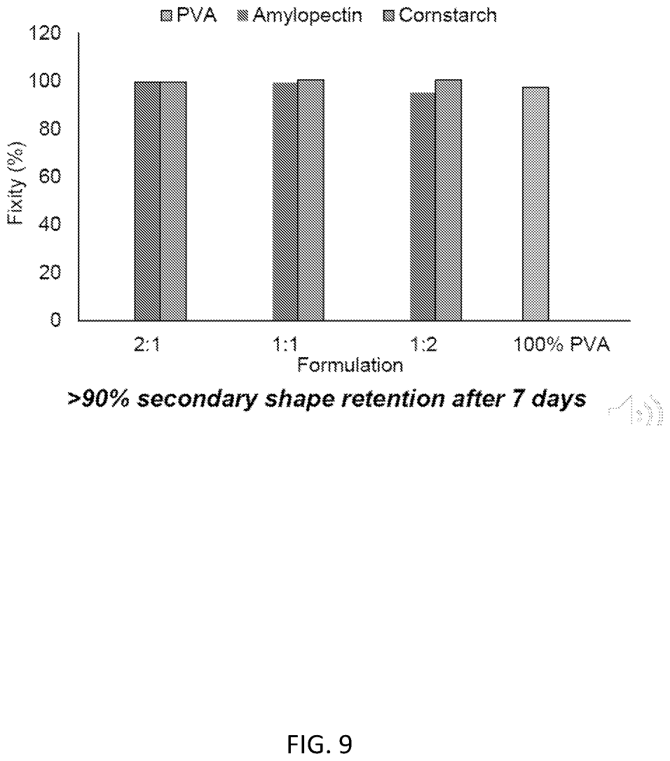

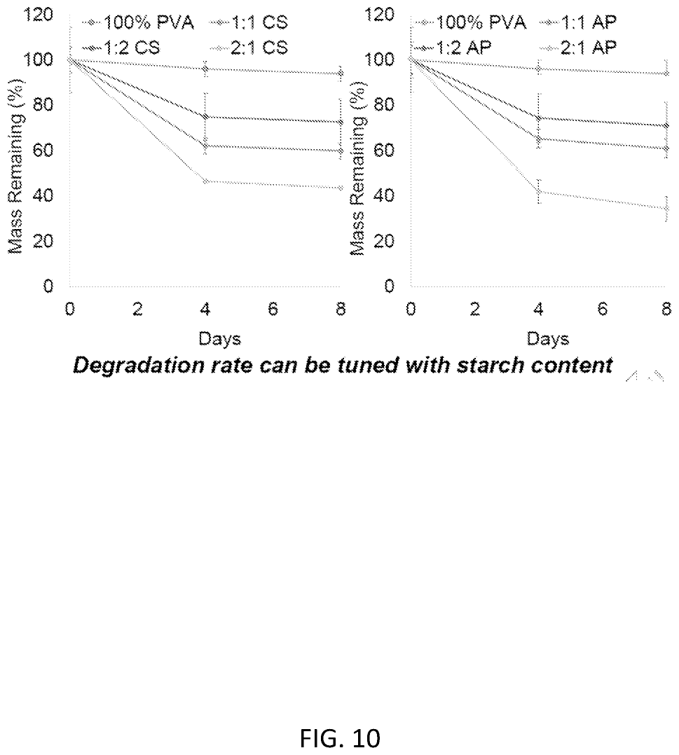

The present invention is also designed break down in response to intestinal epithelial cell (IEC) proliferation and a concomitant increase in starch degrading enzymes during fistula healing. As an example, a series of PVA/cornstarch (CS)-based hydrogel foams were synthesized with varied CS composition. Wet hydrogel foams can be radially crimped and dried to lock in the low-profile geometry, as seen in the top panel of FIG. 3. The relatively low wet compressive modulus enables radial compression into the crimped geometry that is maintained with approximately 100% shape fixity in the relatively high stiffness dry state, as seen in the middle panel of FIG. 3. When the hydrogels are placed in 37° C. water they rapidly expand back to their primary shape within approximately 5 minutes, followed by additional swelling to the equilibrium swelling state over the next 12 hours. As CS content increases in the hydrogels, their enzymatic degradation rate in glucoamylase solution increases, as seen in the bottom panel of FIG. 3. The PVA-CS hydrogel system of the present invention thus provides a platform for cell-responsive enzymatic degradation by encapsulated and/or surrounding cells and with cell-responsive degradation in Crohn's fistulas. For example, various embodiments of the scaffold of the present invention and the characteristics of those embodiments are seen in FIGS. 5 through 11.

### Example 1

In order to incorporate PAs into SMP hydrogels, PVA methacrylate (PVAMA) may be synthesized using an esterification reaction with methacrylic anhydride (MA). Methacrylated cornstarch (starchMA) and gelatin (gelMA) will also be synthesized using reactions with MA. PVAMA will be further modified with varying amounts of PAs with desired functionality using a diisocyanate linker. Briefly, PAs will be dissolved in dimethyl sulfoxide in a 1:1 molar ratio with hexamethylene diisocyanate, forming an amide linkage at the carboxylic acid and a leaving a free isocyanate functional group. The hydroxyl groups on PVAMA will then be reacted with the free isocyanates on the modified PA, providing a PVAMA-PA polymer. The resulting PVAMA-PA will be crosslinked with varying amounts of starchMA and gelMA using UV polymerization with lithium phenyl-2,4,6-trimethylbenzoylphosphinate (LAP, photoinitiator), forming a networked polymer with pendant PAs. The resulting polymer will be characterized in terms of surface chemistry (Fourier transform infrared (FTIR) spectroscopy), compressive mechanical properties, cytocompatibility of IECs and MSCs (Live/Dead assay), gel fraction, swelling ratio, thermal transitions (Tₜᵣₐₙₛ, differential scanning calorimetry (DSC)), and shape memory properties (DMA and volume recovery). Unmodified PVA/starch hydrogels will serve as controls. PVAMA-PA will show an increase in FTIR peaks corresponding to urethane formation and aromatic ring of the PAs. PA-containing hydrogels should have >75% cytocompatibility. Modulus in the dry state should be at least 1 order of magnitude higher than the wet state to allow of good shape memory properties. Gel fractions should be >80% to show good crosslinking efficiency. Swelling ratio should be >100% to ensure hydrophilicity. Tₜᵣₐₙₛ should be above 25° C. in the dry state to enable stable storage in the compressed form and below 37° C. in the wet state to enable expansion after implantation. Hydrogels should have shape fixity and recovery >85% to ensure shape memory properties.

### Example 2

In order to characterize bacteria response to PA-containing hydrogels and antimicrobial properties over time, cylindrical samples may be incubated in PBS at 37° C. for 0, 10, 20, or 30 days. The concentration of PA in PBS will be quantified at each time point using ultraviolet-visible (UV-vis) spectroscopy against a standard for each PA of interest. After incubation, hydrogels will be sterilized via incubation in 70% ethanol overnight and subsequent washing in sterile phosphate buffered saline (PBS, 3 washes). E. coli will be grown overnight in 5 ml of lysogeny broth (LB) at 37° C. Subsequently, 500 μl will be taken from the overnight culture and grown in 10 ml of fresh LB to optical density (O.D.) 0.6 (i.e., until bacteria enter log phase growth). Hydrogel samples will be placed into a 96 well plate, and 100 μl of bacteria solution will be pipetted onto the surface of each sample. Samples will be incubated with bacteria for up to 24 hours at 37° C. and then vortexed to dislodge attached bacteria. Bacterial solutions will be diluted by 10⁶ in fresh LB, and 10 μl will be drop-plated onto LB-agar plates overnight at 37° C. Images will be obtained of each specimen plating area, and colony forming unit (CFU) density will be measured by counting the number of colonies. Hydrogels without PAs will serve as a negative control, and control hydrogels soaked in oxacillin will provide a drug-based control. Control PA, PVAMA-PA, PVAMA, and starchMA, and gelMA solutions will also be used. CFU density after exposure to PA-containing hydrogels should be statistically lower than that of control hydrogels without PAs. Antimicrobial efficacy should be retained for >10 days of storage in PBS.

### Example 3

To characterize EMT response to PA-containing hydrogels, hydrogels may be prepared and incubated in PBS over up to 30 days as described above. EMT will be induced in IECs. Cells will be seeded on the surface of hydrogels, and cell morphology will be observed over up to 1 week. Cells will be stained with antibodies for E-cadherin, fibronectin, vimentin, and Snail-1 at 3, 24, 72, and 168 hours to evaluate the state of the EMT over time. A separate study may be done in which the EMT will be induced at the same time as seeding IECs onto hydrogel surfaces to evaluate the effects of PA-containing hydrogels on preventing EMT induction. Unmodified IECs will be used as cellular controls on each sample, non-PA-containing hydrogels will serve as material controls, and PAs in solution will serve as a positive control for reversing EMT. PA-containing hydrogels should induce a morphological change (rounding, more densely packed) in cells, increase E-cadherin, and decrease fibronectin, vimentin, and Snail-1 to indicate that incorporated PAs reverse the EMT. These changes should be higher at later time points (e.g., 168 hrs) in comparison with PAs in solution, due to increased stability of covalently tethered PAs. The EMT should not be affected on control hydrogels without PAs.

EXAMPLES 1 through 3 provide a biomaterial scaffold that is capable of both reducing bacteria numbers and reversing the EMT while enabling new information about the effects of covalent modification of PAs during scaffold incorporation on their biological activity. This system may be applied to a range of areas where infection (e.g., wound healing, implantable devices) and/or the EMT (e.g., cancer, fibrosis) negatively impact healing. In the long-term, the present invention will provide a new option for Crohn's fistula healing that improves outcomes for patients.

An ideal fistula filling material can be left in place to degrade during healing and make space for new tissue formation. As a result, there is a need to characterize cell-responsive degradation of SMP hydrogels. Starch-based hydrogels should degrade in response to enzymes produced during IEC proliferation to allow natural clearance of the fistula filler during healing, so hydrogel degradation profiles in the presence of enzymes and cells should be evaluated to identify a biomaterial scaffold with a clinically relevant cell-responsive degradation profile.

### Example 4

To evaluate SMP hydrogel degradation profiles in enzymatic media, hydrogel slabs and foams may be fabricated with varied concentrations of methacrylated cornstarch, amylose, or amylopectin relative to PVAMA and gelMA. Foams will be synthesized with low and high concentrations of blowing agents to provide low and high porosity compositions, respectively. Cylindrical samples will be incubated in PBS (real-time hydrolytic), 3% H₂O₂ (real-time oxidative), or 100 U/ml of α-amylase or glucoamylase (real-time enzymatic) degradation media over up to 16 weeks. Each week, samples will be weighed, surface chemistry will be characterized via FTIR, Tg will be measuring using DSC, and surface morphology will be imaged using SEM to evaluate degradation profiles. Degradation media solutions will be serially diluted in cell culture media and placed on top of pre-seeded MSCs and IECs each week to measure degradation byproduct cytocompatibility at 3, 24, and 72 hours. Non-starch containing hydrogels will serve as stable controls. Wells with cell culture media will provide cytocompatible controls. Hydrogels should fully degrade within 6 weeks in the enzymatic media while maintaining stability in the hydrolytic and oxidative media to ensure cell-responsive degradation in a clinically-relevant time frame, based on wound healing rates. Higher starch concentrations and increased porosities should increase degradation rates. Cornstarch will likely have an intermediate degradation rate relative to amylose (slowest) and amylopectin (fastest) based on degradable linkages in each starch. Degradation byproducts should have >75% cell viability throughout the degradation process.

### Example 5

To characterize hydrogel degradation in indirect contact with cells, hydrogels may be fabricated with varied porosity and starch content as described above and placed into Transwell inserts above pre-seeded MSCs, IECs, or IECs that have undergone the EMT. Cell viability will be characterized at set time points over 1 week of culture, and hydrogel mass, surface chemistry, Tg, and surface morphology will be characterized every 3 days. To enable longer-term characterization, Transwell inserts will be moved to well plates with fresh cells each week, and degradation profiles will be characterized over up to 6 weeks. Hydrogels will be placed in cell culture media without cells as a media control, non-porous hydrogel slabs will be utilized to characterize effects of porosity on degradation, and non-starch containing hydrogels will serve as biostable controls. Hydrogel degradation rates should be slightly slower than those in EXAMPLE 5, due to the lack of direct contacts between cells and the materials, but trends between formulations should be similar. Degradation in the presence of MSCs and EMT-IECs should be slower than that in the presence of IECs due to reduced enzyme concentrations.¹⁶ Cells should remain viable (>75%) over 1 week of culture in the presence of degrading hydrogels.

### Example 6

CS, hexamethylene diisocyanate (HDI), dimethyl sulfoxide, sodium chloride (NaCl), and DTT were all purchased from Fisher Scientific. 2-Hydroxyethyl disulfide (HEDS) was purchased from TCI Chemicals (Portland, OR). PVA of two different molecular weight (25,000 and 6000 Da) was purchased from Polysciences (Warrington, PA). All cell culture solutions and chemical were purchased from ThermoFisher (Waltham, MA).

A polyurethane DSX was synthesized using HEDS and HDI. Monomers were placed into a round bottom flask in a humidity-controlled glove box at a molar ratio of 2:1 (HDI:HEDS). DMSO dried over molecular sieves was added to the round bottom flask in a mass (g) to volume (ml) ratio of 0.73:1 (monomers:DMSO). The reaction was maintained at 50° C. with constant stirring under nitrogen until Fourier transform infrared (FTIR) spectroscopy confirmed a complete reduction of hydroxyl peak (‘3200-3500 cm⁻¹). DSX was then stored within the glove box for further use.

Previously weighed CS and PVA were dissolved in DMSO (dried over molecular sieves) at 90° C. under nitrogen to make a 10% solution (w/v). Once completely dissolved, the solutions were allowed to cool to room temperature. DSX was added to a speed mixing cup inside the glove box. The PVA/CS mixtures were then combined in varying ratios with the DSX outside of the glove box. The DSX:polyol weight ratio was 0.364:1. The hydrogel components were mixed using a Flacktek high speed mixer (Landrum, SC) at 3500 rpm for 10 s. The contents were poured into a glass petri dish and crosslinked at 50° C. for 8 h. Upon completion of crosslinking, films were washed twice in deionized (DI) water for 20 min to remove any unreacted material and residual DMSO. Synthesized hydrogel formulations are shown in Table 1. Samples were air dried overnight and then further dried in a 50° C. vacuum oven for 24 h. Samples were examined by FTIR after drying.

Foams were synthesized using the same protocol as the hydrogel films with the addition of vacuum dried NaCl porogens (sieved to 300-500 m; weight ratio of 32 NaCl:1 hydrogel components) to the speed mixer cup in the mixing step. After mixing, samples were vortexed for 30 s, and foams were polymerized in the speed mixer cup. Foams were placed into DI water for 24 h on a 37° C. shaker table at 100 rpm to remove NaCl porogens, and then vacuum dried for 24 h at 50: C. Samples were examined by FTIR after drying

Prior to washing films with DI water, 8 mm biopsy punches were taken (n=3). Samples were dried in a 50C vacuum oven for 24 h. Samples were then weighed (Wi) and placed into a vial containing DI water. The vials were incubated at 50C for 24 h. DI water was removed, and film pieces were vacuum dried at 50° C. for 24 h. The samples were then reweighed (Wf). Gel fraction was calculated.

TABLE 1
Synthesized hydrogel formulations with varying polyvinyl alcohol
(PVA):cornstarch (CS) ratios and PVA molecular weights

| Sample | PVA MW (kDa) | PVA ratio (%) | CS ratio (%) |
|---|---|---|---|
| 0:1 | 6 | 100 | 0 |
| | 25 | 100 | 0 |
| 1:2 | 6 | 67 | 33 |
| | 25 | 67 | 33 |
| 1:1 | 6 | 50 | 50 |
| | 25 | 50 | 50 |
| 2:1 | 6 | 33 | 67 |
| | 25 | 33 | 67 |

Density of films (Df) and foams (Dₚ) were measured on 6 mm biopsy punches using mass and volume measurements (n=3).

Thin hydrogel discs were collected from casted films using a 6 mm biopsy punch. Film samples (n=3) were weighed in the initial dry state (Wi) then placed into vials containing 37° C. DI water. Vials were incubated in a water bath at 37° C. to maintain the temperature. Samples were removed from the vials and weighed (Wt) at 1, 5, 10, 30, and 60 min and at 24 h. Swelling ratio was calculated using

Foam slices (n=3) for each formulation were cut and sputter coated with gold for 45 s. Foams were imaged using Jeol NeoScope JCM-5000 Scanning Electron Microscope (SEM). Pore size was determined by analyzing 50 total pores per formulation using the measuring tool in ImageJ. Pores size was reported as the average diameter of measured pores

Caco-2 cells were cultured in Dulbecco's modified Eagle's medium supplemented with 10% fetal bovine serum and 1% Penicillin/Streptomycin (Pen/Strep). Cells were seeded into 24 well plates at a density of 5000 cells per well 1 day prior to testing. Samples (n=3) weighing 10 mg (dry) were sterilized by incubating in 70% ethanol overnight and then moving into sterile phosphate buffered saline (PBS) for 3 h before placing directly into wells. Cytocompatibility was measured at 24, 72, and 168 h using a resazurin assay, in which a 10% resazurin solution (prepared in cell culture media) was added to cells and incubated for 1 h. Using a plate reader, fluorescence absorbances (excitation 530 nm and 590 emission) were obtained and compared to an untreated control. Cytocompatibility was calculated by

Film (n=3) and foam (n=3) samples were characterized in terms of tensile and compressive properties, respectively (Model 100P Universal Testing Machine, Test Resources, Shakopee, MN). Films were swollen in 37° C. PBS and cut into dog bones with gauge length of 6.25 mm and width of 1.5 mm, which were stretched at a rate 5 mm/min until failure. Cylindrical foam specimens (7 mm diameter and 3.5 mm height) were compressed in the dry and wet states at 1 mm/min. Wet samples were tested directly after removal from PBS.

TABLE 2
Characterization summary of synthesized hydrogels

| Formulation | 0 CS:1 PVA | | 1 CS:2 PVA | | 1 CS:1 PVA | | 2 CS:1 PVA | |
|---|---|---|---|---|---|---|---|---|
| PVA Mw | 6k | 25k | 6k | 25k | 6k | 25k | 6k | 25k |
| Gel | 87 ± | 77 ± | 84 ± | 80 ± | 78 ± | 71 ± | 86 ± | 73 ± |
| fraction (%) | 1 | 1 | 1 | 3 | 1 | 2 | 2 | 2 |
| 24 h swelling | 1.06 ± | 1.04 ± | 1.07 ± | 1.03 ± | 1.19 ± | 1.17 ± | 1.18 ± | 1.18 ± |
| ratio | 0.01 | 0.02 | 0.04 | 0.11 | 0.12 | 0.06 | 0.06 | 0.10 |
| Pore size | 200 ± | 220 ± | 180 ± | 210 ± | 240 ± | 200 ± | 190 ± | 210 ± |
| (μm) | 40 | 40 | 40 | 40 | 60 | 60 | 50 | 30 |
| Porosity | 57 ± | 71 ± | 63 ± | 73 ± | 67 ± | 73 ± | 57 ± | 71 ± |
| (%) | 9 | 1 | 5 | 4 | 2 | 6 | 7 | 2 |

Differential scanning ca orimetry (DSC was use to examine thermal properties. Foam samples (n=3) weighing 3-5 mg were placed into aluminum T-Zero pans. Samples were subjected to the following thermal cycle: equilibration at 0° C. for 2 min, heated to 180° C. at 10° C./ min, held isothermally for 2 min, cooled to 0° C. at 10° C./min, held iso-thermally for 2 min, and heated to 180° C. at 10° C./min. Thermal transitions were taken from the second heating cycle.

Foams were placed in water for 1 h and then cut into cylindrical samples (6 mm diameter, 4 mm height, n=3). After cutting, samples were radially compressed using a BlockWise radial crimper (Tempe, AZ). The crimper was then placed into a room temperature vacuum oven for 24 h to dry the samples in the compressed state. Upon removal, radius was measured (Ri) and samples were incubated at room tem-perature and ambient humidity for 24 h, after which samples radius was measured again (Rf). Shape fixity was calculated by:

$$Shape fixity = \frac{\text{?} - ( \text{?} - \text{?} )}{\text{?}} \times 100.$$

$$\text{?} \text{indicates text missing or illegible when filed}$$

Samples were then submerged in 37° C. water, and images were taken every 10 s for 30 min. At 30 min, samples were removed, and diameter was measured. Using ImageJ, sample diameter (Rt) was quantified at each time point by calibrating to the 60-min water exposure length and the initial diameter (Rf). Shape recovery was calculated as

$$Shape recovery = \frac{\text{?}}{\text{?}} \times 100.$$

$$\text{?} \text{indicates text missing or illegible when filed}$$

Dry samples of each formulation (n=3) weighing between 20 and 30 mg were swollen in PBS for 24 h prior to starting degradation. Samples were weighed and placed in 5 ml of PBS (control media), 10 mM DTT (disulfide degrading media), 100 U/ml amylase (CS degrading media, or 10 mM DTT+100 U/ml amylase. All degradation solutions were prepared in PBS. Media was changed daily. Samples were weighed every 2 days for up to 20 days after lightly pressing dry for 10 s using a paper towel. Swollen mass remaining was calculated by:

$$Swollen mass remaining ( \% ) = \frac{Mas \text{?}}{Mas \text{?}} \times 100 \% .$$

$$\text{?} \text{indicates text missing or illegible when filed}$$

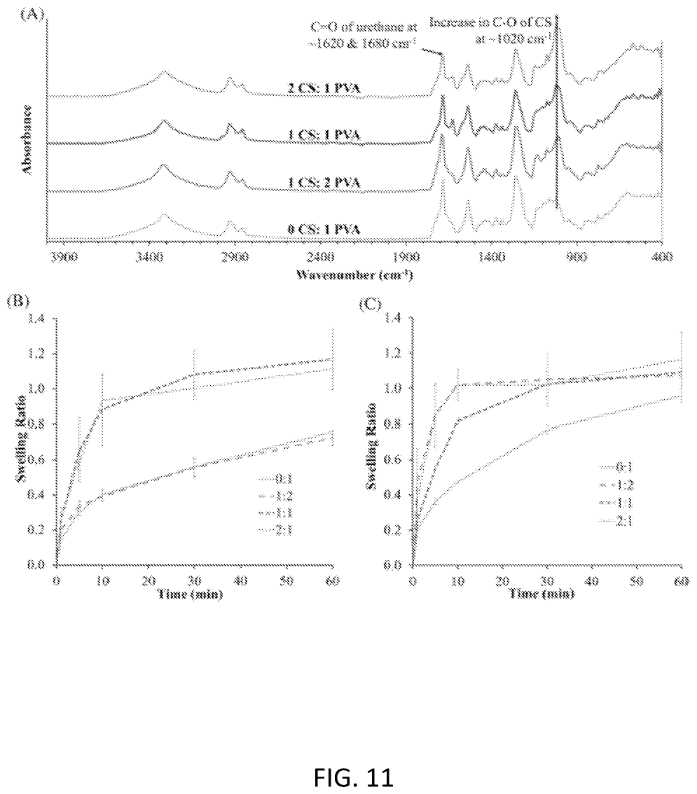

Measurements are presented as mean±standard deviation. Student's t-tests were performed to determine differences between formulations, where a p-value of <0.05 was taken as statistically significant. The FTIR spectra of CS/PVA hydrogels (FIG. 11A) showed a peak at ^{⋅}1680 cm⁻¹ in all formulations. This peak is associated with the urethane linkage formed between HEDS and HDI in the DSX. An increased absorbance at ^{⋅}1020 cm⁻¹ was observed with increasing CS content. This peak is attributed to the glycosidic ester between starch molecules (C—O—C). In addition, with increasing CS content, a peak at ^{⋅}1620 cm⁻¹ emerges that corresponds to the urethane linkage between the DSX and CS. These spectra confirm successful synthesis of crosslinked polyurethane-based PVA/CS hydrogels. FIG. 11A shows the PVA 6 k formulations; similar trends were observed in the PVA 25 k hydrogel FTIR spectra.

In general, gel fractions in the PVA 6 k formulations were higher than those in the PVA 25 k hydrogels and were in the range of 78%-87% (vs. 71%-80% for PVA 25 k). Hydrogels with 1:1 CS:PVA ratios had the lowest gel fraction in both sets of hydrogels. Overall, these relatively high gel fractions demonstrate successful crosslinking of PVA/CS hydrogels with the DSX.

Equilibrium swelling ratios of hydrogel films at 24 h generally increased with increased CS content in both hydrogel sets, Table 2. The molecular weight of PVA did not significantly affect swelling in any of the testing groups. Within the PVA 6 k formulations (FIG. 11B), those with lower CS concentrations had a slower swelling rate before achieving equilibrium. For PVA 25 k hydrogels (FIG. 11C), the 0:1 formulation with no CS showed the slowest swelling rate.

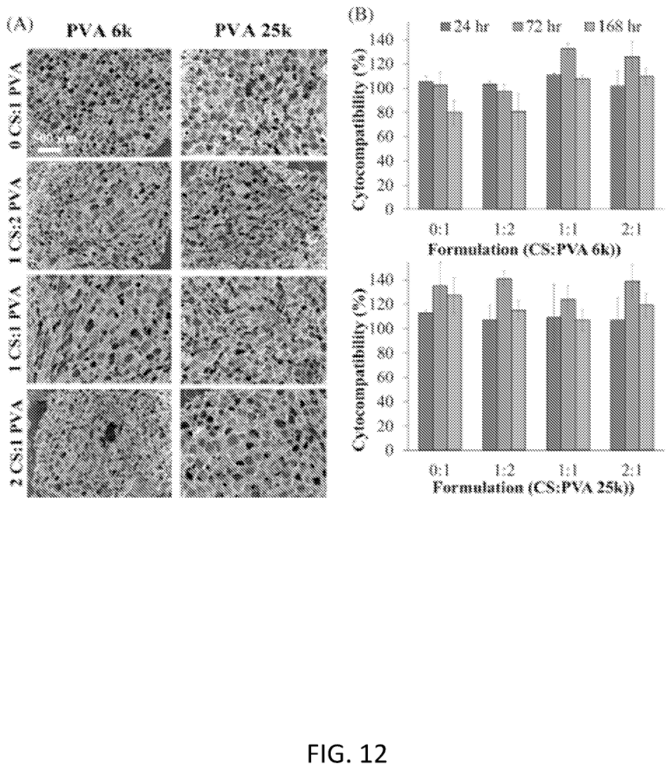

Foam pore sizes were slightly smaller on average in the majority of the PVA 6 k hydrogels, but no statistically different pore sizes were measured between the formulations. Porosity was also generally lower in the PVA 6 k formulations in comparison with the PVA 25 k hydrogels. Within a given PVA molecular weight, no significant differences in porosity were measured, and no clear trends were observed based on CS content. Uniform pore shape and distribution throughout the scaffolds was observed in SEM images of the foam formulations (FIG. 12A).

All formulations showed Caco-2 cell cytocompatibility >80% at 24, 72, and 168 h, FIG. 12B. Samples containing PVA 25 k showed slightly higher cytocompatibility, with all formulations having >100% cytocompatibility in comparison with empty control wells at 24 h.

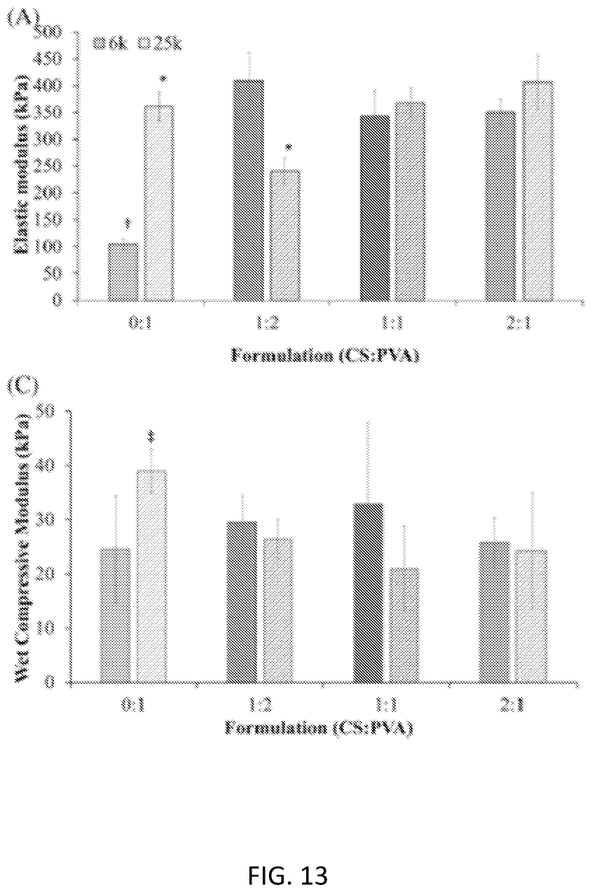

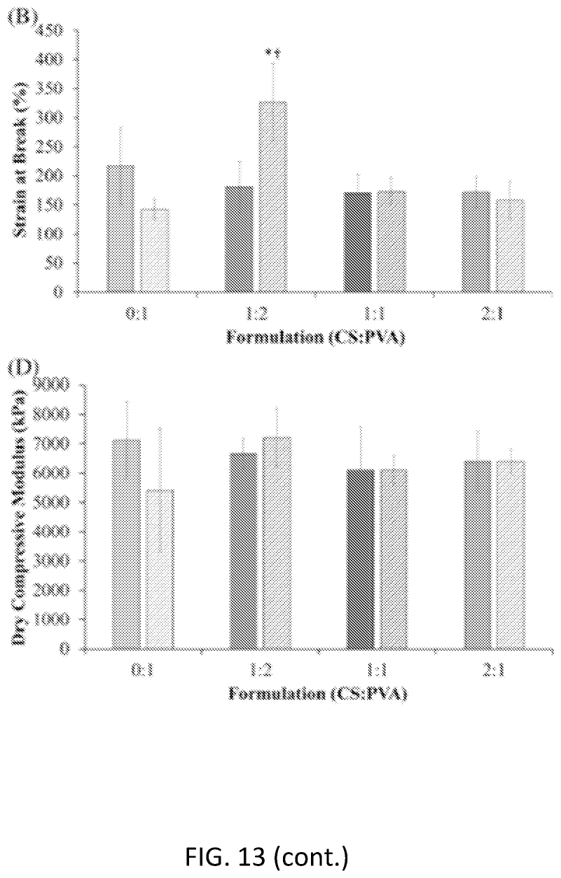

Elastic modulus measurements of 6 k hydrogel films showed that with the addition of CS, the material becomes significantly stiffer (FIG. 13A). The 0:1 PVA 6 k hydrogels had the lowest elastic modulus of all the formulations (105 kPa, significantly lower than PVA 25 k 0:1 hydrogel), with all other formulations ranging between ′340 and 400 kPa. Hydrogels synthesized with PVA 25 k generally had fewer modulus variations with the addition of CS. Strain at break was similar between all formulations regardless of CS content and PVA molecular weight, with the exception of the 1:2 PVA 25 k hydrogel, which had a significantly higher strain at break relative to the corollary PVA 6 k hydrogel and relative to all other PVA 25 k hydrogels. This result may be due to the packing structure of the polymer chains. The addition of a lower concentration of CS may have disrupted the PVA network organization, reducing overall stiffness and increasing elongation. Compressive modulus of porous hydrogel foams was generally similar between all testing groups in both the wet and dry states, FIG. 13C,D. The highest wet compressive modulus was recorded in the 0:1 foams containing PVA 25 k. Dry compressive moduli for all the formulations were significantly higher (>2 orders of magnitude) than in the wet state.

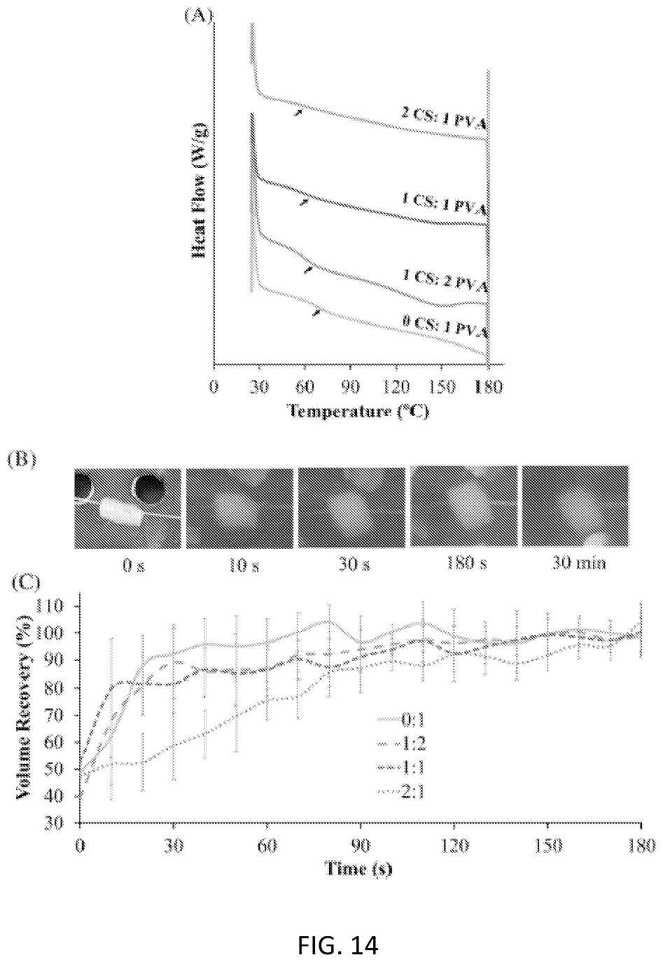

Hydrogel foams containing PVA 6 k showed glass transition temperatures (T_{g}'s) at ′73° C. in DSC thermograms. When the molecular weight of PVA was changed to 6 k, T_{g} dropped to ′62° C., except for the 0:1 PVA 6 k foam, which had a T_{g} of 71° C., FIG. 14A. In addition, a shallowing of the T_{g} step was observed with increasing starch content.

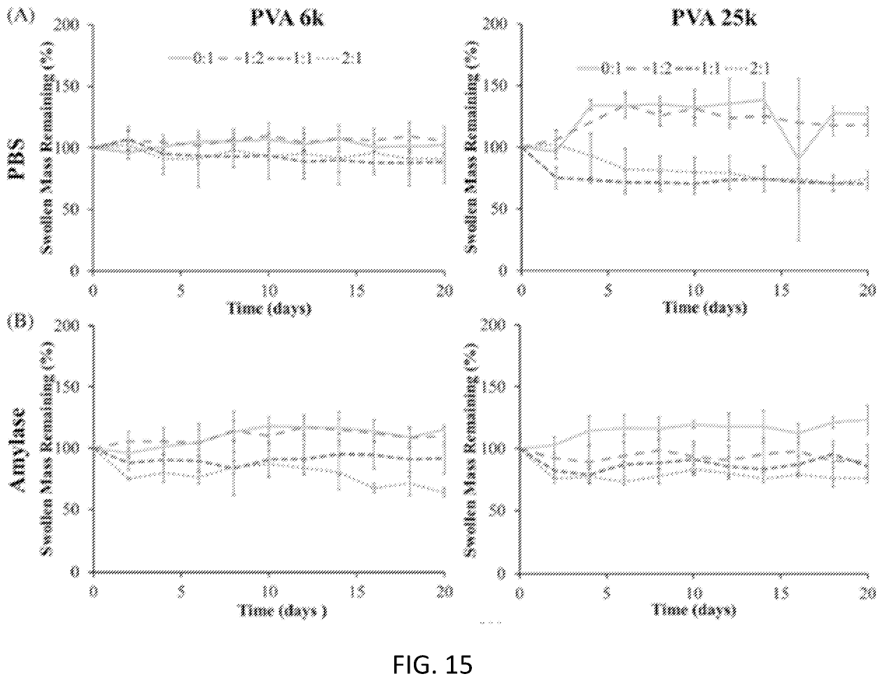

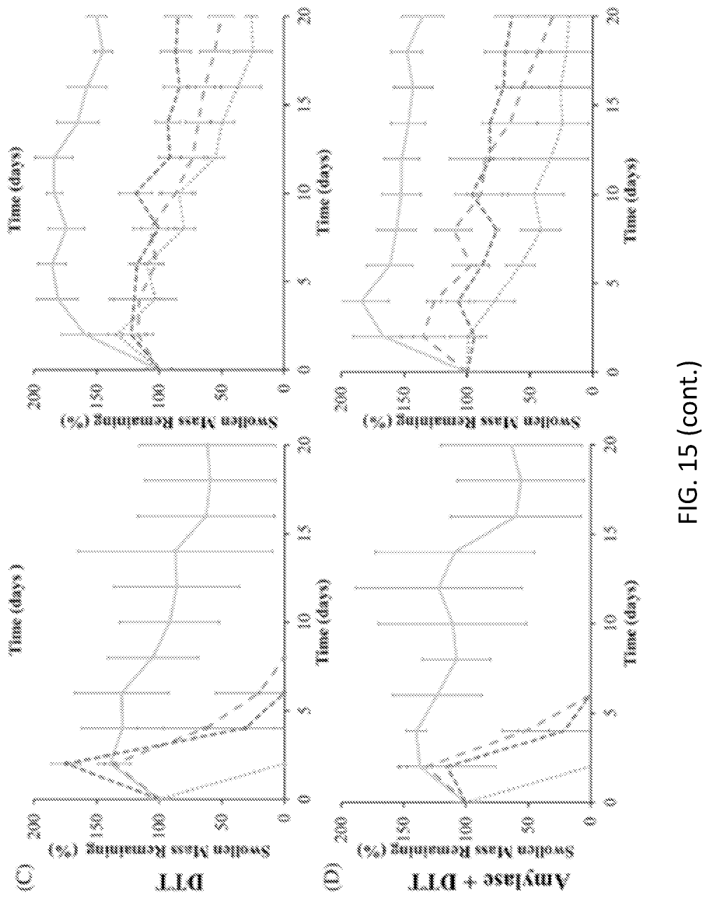

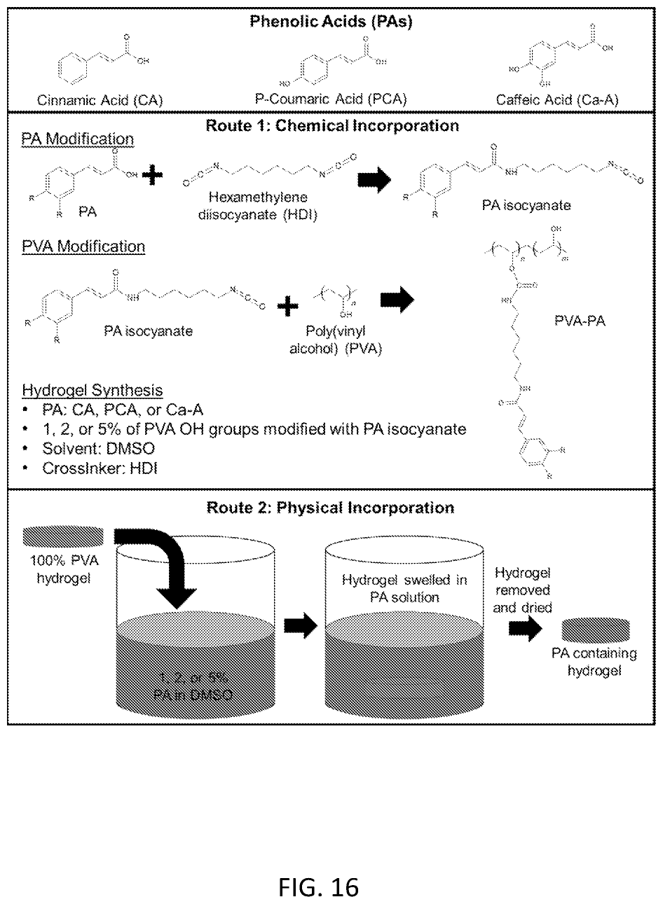

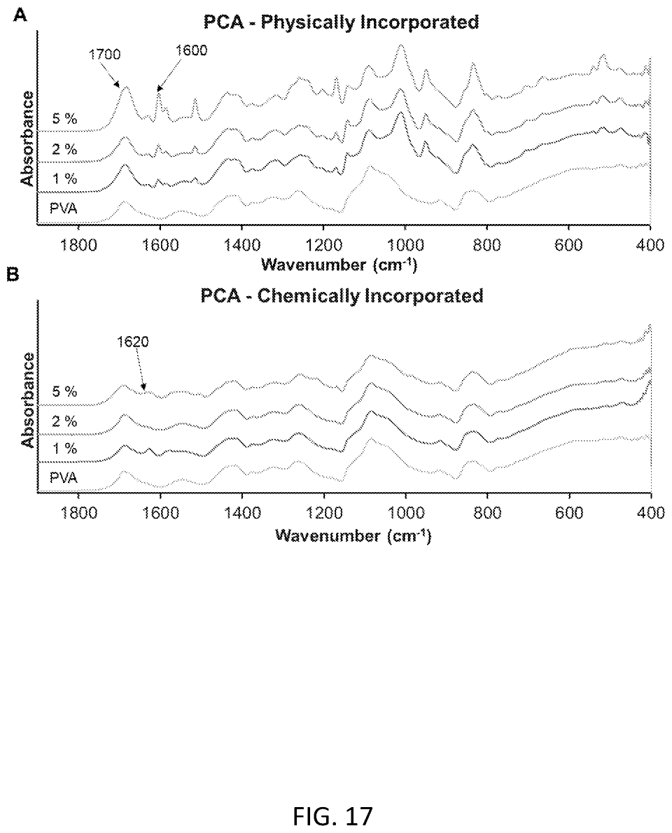

All foams had high retention of the compressed shape after 24 h, with shape fixity values between 97% and 99%. Additionally, all foam formulations showed rapid shape recovery when placed into 37° C. water. Complete shape recovery to the initial wet diameter was achieved in all formulations within 180 s, FIG. 15B,C. In general, shape recovery was slower with increased CS content.

Phosphate buffered saline was employed as a control degradation media base in these studies to enable comparison with other in vitro degradation studies in the literature and to provide an understanding of the individual effects of amylase and DTT on hydrogel degradation. No large changes in mass were seen in PVA 6 k formulations in PBS over 20 days (FIG. 15A, left). There were some visible changes in the mass of the PVA 25 k hydrogels in PBS (FIG. 15A, right). Namely, formulations containing a higher amount of PVA 25 k had an increase in swelling up to ′130% after 4 days in PBS, while higher CS content foams had a decrease in swollen mass down to ′75% after 4 days.

In amylase, which can degrade CS, a general increase in mass loss was observed in higher CS content hydrogels for both PVA molecular weights, FIG. 15B. When hydrogels were treated with DTT, which degrades the DSX, the PVA 6 k foams underwent faster degradation than PVA 25 k foams, FIG. 15C. The PVA 6 k foams that contained CS underwent complete degradation within 2-8 days in DTT, with the fastest degradation observed in the highest CS content foam (2:1). The 0:1 PVA 6 k control had an initial increase in swollen mass followed by a steady decrease to 62% mass at 20 days. In 25 k PVA formulations, the 0:1 PVA control had an increase in swelling that plateaued after 4 days. In the CS containing PVA 25 k foams, there was a steady loss of mass observed for all formulations, with the slowest mass loss observed in the 1:1 formulation and faster degradation in the 1:2 and 2:1

In both PVA 6 k and 25 k foams, combined treatment with DTT and amylase resulted in similar mass loss trends as seen with DTT alone with faster overall mass losses in the CS-containing foams, FIG. 15D. The 0:1 PVA control mass loss profiles were comparable to those obtained in DTT alone. All CS-containing PVA 6 k foams had complete mass loss within 6 days, with the fastest degradation again observed in the 2:1 PVA 6 k foam at 2 days. All CS-containing PVA 25 k formulations underwent higher amounts of mass loss in the combined media (19% vs. 26% swollen mass at 20 days for 2:1 foam; 32% vs. 50% swollen mass at 20 days for 1:2 foam; and 64% vs. 85% swollen mass at 20 days for 1:1 foam).

Using simple polyurethane-based chemistries, it is possible to achieve a SMP hydrogel with high tunability and dual biodegradation mechanisms. Previously PVA has been crosslinked into layered membranes using an isocyanate DSX for islet encapsulation and for self-healing poly-mers. CS-based disulfide materials have also been used to develop nanoparticles for drug delivery. The synthesis method differs from previously published work on disulfide reductive polymers, in that DSXs are typically based on acidic disulfides, such as dithiodipropionic acid. Other disulfide crosslinking methods include isocyanate-terminated prepolymers synthesized using materials without pendant OH groups, such as PEG or PCL (i.e., only terminal OH groups), which were then crosslinked using a disulfide-containing polyol. In this approach, the disulfide linkages are only located at the ends of prepolymer chains, limiting the overall tunability of the materials. Additionally, using PCL in the crosslinker can have a negative effect on hydrophilicity, swelling, and mechanical properties. The method developed here allows for an easy, single-step synthesis of a non-acidic DSX that can be employed with a range of isocyanate-reactive materials, including polyols, such as CS and PVA used here. Using FTIR and gel fraction measurements, it was confirmed that this approach successfully crosslinks CS and PVA into a disulfide-containing polyurethane network.

The lower molecular weight PVA 6 k hydrogels fabricated using this method generally had higher gel fractions than the PVA 25 k gels.

This result may be due to higher mobility of smaller PVA chains, which enables increased interactions with free isocya-nates during crosslinking. A similar trend was previously reported in PVA hydrogels crosslinked with glutaraldehyde. Higher gel fractions in the PVA 6 k gels correlate with lower porosity values, and the pore size and morphology (FIG. 12A) was similar between all the formulations. The density of PVA 6 k hydrogels is higher due to increased gel fractions, which reduces overall porosity. Porosity can easily be adjusted by adding more salt particles and/or increasing the size of salts used in the porogen leaching process.

The slight increase in swelling seen with higher CS content can be attributed to the effect that CS has on polymer chain packing. More bulky ring structures and branching increases space between polymer chains. A previous study show that a glutaraldehyde-crosslinked PVA starch composite had a slightly higher swelling once the composite was ≥50% starch. Additionally, more rapid swelling was observed with increasing CS content, which also correlates with previous reports.

The tensile modulus of the bulk hydrogel films is very similar to native tissue (FIG. 14); colon tissue has a reported elastic modulus between 300-800 kPa. Additionally, the hydrogels show highly elastic behavior with elongation at break of 150%. This value is 2.5-3 times larger than that reported in native tissue. This bio-material system has an overall advantage of high synthetic tunability, allowing us to control the modulus of our materials in future work while better matching native tissue elasticity. For example, by changing the type of isocyanate to one with a more bulky structure, such as methylene diphenyl diisocyanate, or by increasing the crosslink density with a higher DSX content, it is possible to easily tune the modulus of our material.

In general, using porous biomaterials allows for ingrowth of host tissue and increased nutrient transport, with the tradeoff of reduced stiffness with the introduction of pores. A dry compressive modulus that is >2 orders of magnitude higher than the wet modulus in this system enables a water-responsive SMP that changes shape after exposure to body temperature water. This property allows us to program a low-profile, compressed foam shape in the wet state and dry the material for high shape fixity (>97%). Theoretically, this material could be delivered via catheter in the compressed form. The quick recovery (within 3 min) can be employed during implantation, so that the material expands after delivery to fill the fistula site. Current natural bioprosthetics that are used in fistula treatment must be pre-swollen before implantation, which increases overall procedure time. One limitation to water-based shape memory is the long drying time required for fixing. Further studies on the effects of high heat drying will be carried out to optimize translation of these materials. While the water-responsive SMPs do show consistent thermal transitions (Tg's) there is not enough modulus variability above and below the T_{g} to allow for traditional, thermally-induced dry shape programing.

Degradation of these materials was tuned with both PVA molecular weight and CS content. Polymers containing PVA 6 k showed faster degradation compared to PVA 25 k hydrogels in DTT-containing media. While the molecular weight of PVA was increased in these scaffolds, the number of available hydroxyls for crosslinking was held constant. This property is reflected in the relatively consistent mechanical properties between PVA 6 k and 25 hydrogels. Thus, the number of crosslinks/per chain leads to a difference in degradation rate. Lower molecular weight PVA will have fewer crosslinks per chain, which will allow for chains to be more easily released from the network. This finding allows for even more tools to tune the degradation rate independently of mechanical properties in future work through the use of additional PVA molecular weights.

In the PVA 6 k hydrogels, degradation rates in amylase and/or DTT were consistently increased with increased CS content. However, in the PVA 25 k foam in DTT (with or without amylase), CS content at unequal ratios relative to PVA (1:2 and 2:1) led to faster degradation compared with the 1:1 CS:PVA 25 k. This result is attributed to the organization of the polymer structure that allows for easier penetration of DTT through more consistent networks.

Amylase is produced by intestinal epithelial cells, and glutathione is a reducing agent that is abundant in the intestinal mucous. Thus, these hydrogels have mechanisms for potential degradation by amylase (CS) and by reducing agents (DTX) after implantation into a fistula site. Amylase alone can partially degrade the foams, but degradation quickly reaches a plateau, which is likely the point at which most of the CS has been broken down, leaving behind a PVA network. This property could enable a tunable drug release mechanism in this system in future work. Here, DTT was used as a representative reducing agent to evaluate the ability to degrade the DTX in the hydrogels. DTT was effective at fully degrading the hydrogels, which was expected, as the crosslinkers were degraded to leave behind soluble PVA and CS. Increasing CS content increased degradation rates in DTT, which may be due to increased water interactions (evidenced by faster swelling profiles and increased equilibrium swelling ratios). Combined treatment with DTT and amylase increased the degradation rate in the CS-containing hydrogels, while the 0:1 PVA control hydrogels had similar degradation profiles in DTT regardless of amylase addition. Again, this result is expected since amylase should not affect the PVA backbone.

While this study provides a foundational understanding of the individual attributes of this material platform, DTT is not naturally found in the human body. Additionally, 10 mM DTT provides a much higher concentration than that of reducing agents that would typically be found at in human tissue, as serum levels of reducing-capable thiols range from 0.35 to 0.55 mM. Therefore, the measured degradation profiles in DTT are accelerated to provide a big picture understanding of hydrogel structure effects on degradation properties, and real-time degradation must be assessed using physiologically-relevant concentrations of using glutathione.

### Example 7

This Example relates to chemical and physical incorporation approaches to modify crosslinked PVA-based polyurethane hydrogels with three cinnamic acid-based PAs (cinnamic (CA), p-coumaric (PCA), and caffeic (Ca-A) acid). The effects of modification on mechanical and shape memory properties, cytocompatibility, PA release, and antimicrobial efficacy were evaluated. The shape memory properties of these materials allow for rapid fistula filling while PAs provide local antimicrobial properties. Additionally, the inclusion of PAs can be used to tune intermolecular interactions between polymer chains to alter material mechanical and shape memory properties.

Hexamethylene diisocyanate (HDI) was purchased from TCI chemicals. Polyvinyl alcohol (PVA, 25,000 MW) was purchased from Polysciences. Cinnamic acid (CA), p-coumaric acid (PCA), and caffeic acid (Ca-A) were purchased from TCI chemicals. Dimethyl sulfoxide (DMSO) and phosphate buffered saline (PBS) tablets were purchased from Fisher Scientific.

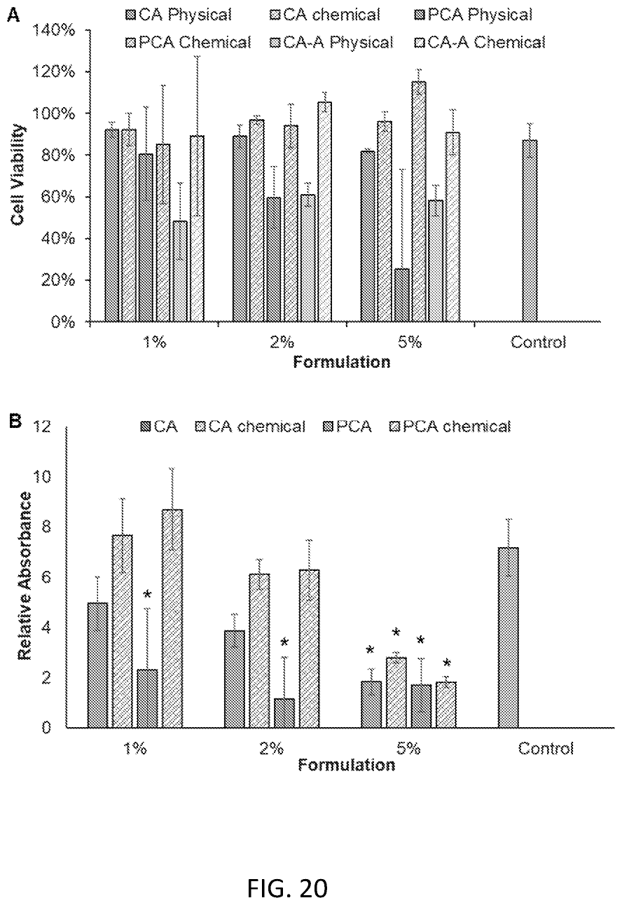

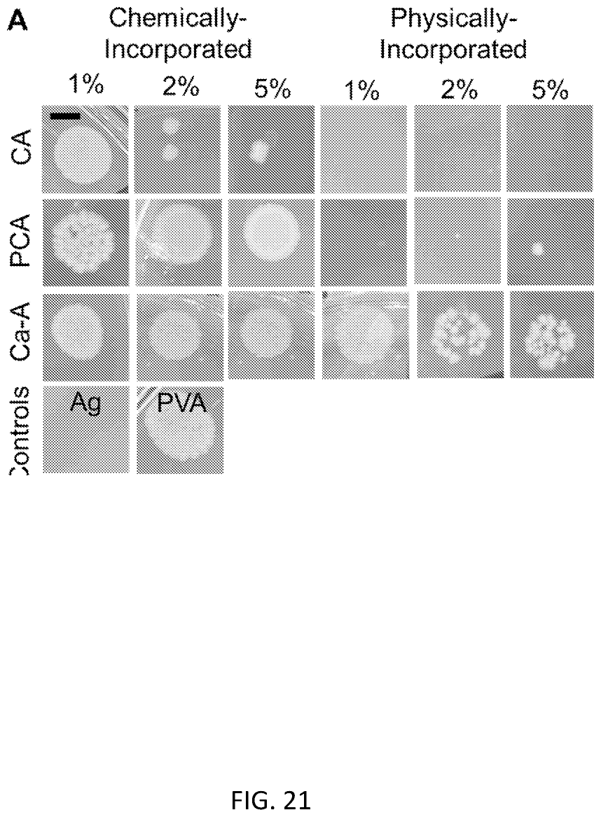

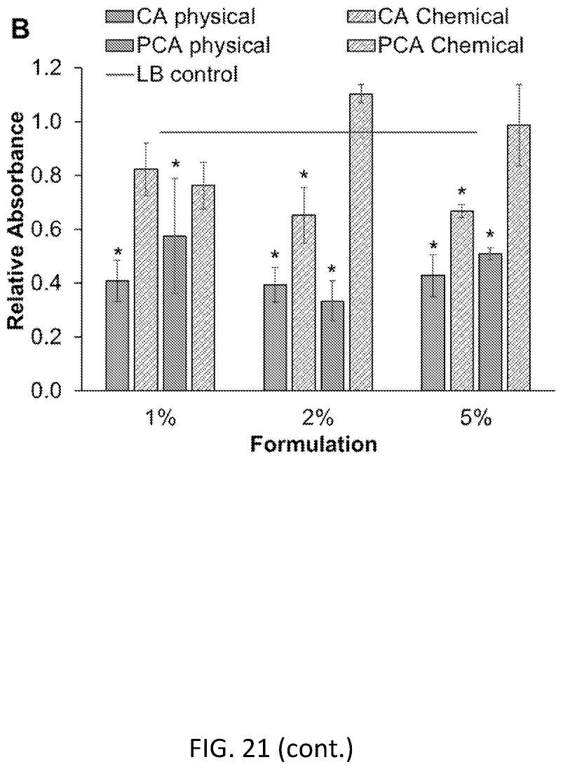

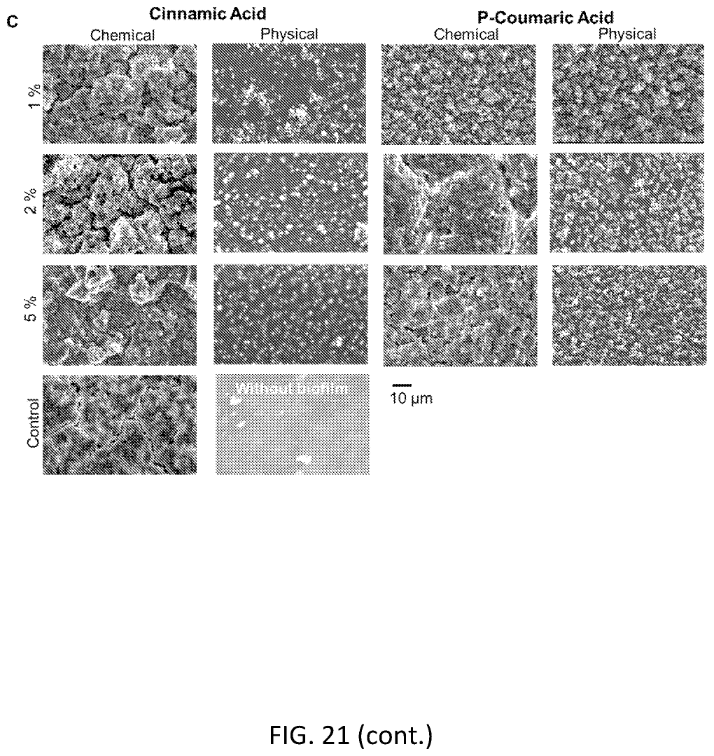

PA-modified films were made by first reacting PAs with hexamethylene diisocyanate (HDI) in DMSO to form PA isocyanates, as seen in FIG. 20. PAs (CA, PCA, Ca-A) were dissolved in sieve-dried DMSO in a Flacktek speed mixer cup. HDI was then added inside a moisture-controlled glovebox (Labconco) to react at a 1:1 molar ratio with the PAs. The cup contents were mixed using a Flacktek high-speed mixer at 3500 rpm for 15 seconds and placed in a 50° C. oven to react for 24 hours. PVA was then dissolved in sieve-dried DMSO in a round bottom flask at 90° C. at 100 mg/ml. When completely dissolved, the solution was cooled to room temperature. HDI was added (0.033 mol. eq. relative to PVA OH) to the PA isocyanates (1, 2, or 5 mol. eq. relative to PVA OH) in the glovebox and placed in a high-speed mixer at 3500 rpm for 15 seconds. Dissolved PVA was then added to the speed mixer cup and mixed for an additional 15 seconds at 3500 rpm. The cup contents were poured into a glass petri dish and allowed to cure overnight at 50° C. Resulting hydrogel films were washed twice in deionized (DI) water for 20 minutes each to remove any residual DMSO. Samples were dried in a 50° C. vacuum oven for 24 hours and, upon completion of drying, were examined with Fourier transform infrared spectroscopy.

PVA was dissolved in DMSO at 90° C. at 100 mg/ml. The mixture was cooled to room temperature. HDI was added to the PVA in the glovebox and the solution was placed in the high-speed mixer at 3500 rpm for 10 seconds. Contents were poured into a glass petri dish and cured overnight at 50° C. Films were washed twice in DI water for 20 minutes each. PA solutions were prepared at 1, 2, and 5 wt % in DMSO. PVA film samples (6 mm biopsy punches and tensile dog bones) were incubated in these solutions at 50° C. for 24 hours. Following physical incorporation, sample pieces were dried in the 50° C. vacuum oven to remove DMSO and then characterized.

Gel fractions were measured on chemically incorporated PA-PVA hydrogels only, as PAs would be released from physically incorporated PA-PVA hydrogels to alter results. Before washing films with DI water, 6 mm biopsy punches were taken (n=3). Samples were dried in a 50° C. vacuum oven for 24 hours. Upon drying, initial weight (Wi) was obtained. Samples were placed in a vial and swelled in DMSO. Vials were incubated at 50° C. for 24 hours. DMSO was removed and samples were vacuum dried at 50° C. for 24 hours. Film pieces were reweighed to obtain dry weight (W_{d}). Gel fraction was calculated as:

$$Gel fraction ( \% ) = \frac{{W}_{d}}{{W}_{i}} * 100$$

Swelling ratios were obtained on chemically incorporated PA-PVA hydrogels. Following the recording of the dry weight (W_{d}), samples were placed in vials and swelled in 37° C. deionized (DI) water. Vials were placed on a 37° C. orbital shaker to maintain temperature for 24 hours. Samples were removed from the vial and weighed (Wₛ). The swelling ratio was calculated as:

$$Swelling ratio = \frac{{W}_{s} - {W}_{d}}{{W}_{d}}$$

Dried PVA hydrogels were weighed (W_{c}) before physical incorporation (n=3). After the completion of physical incorporation, hydrogels were dried under vacuum and reweighed (W_{L}). PA content was calculated as a percent of the hydrogel mass by:

$$PA content ( \% ) = \frac{{W}_{L} - {W}_{C}}{{W}_{C}} * 100 \%$$

Film samples for both chemically (n=3) and physically (n=3) incorporated gels were characterized in terms of tensile properties. Dog bone specimens were placed in a vial of DI water at 37° C. for 1 hour to hydrate before testing. Dog bones had a length of 6.25 mm and a width of 1.5 mm. Samples were stretched at a rate of 5 mm/minute until failure.

Release rates of PAs from chemically (n=3) and physically (n=3) incorporated hydrogels were examined using UV-vis spectroscopy. Calibration curves for CA, PCA, and Ca-A were made using a serial dilution of each PA in a 1:1 mixture of DMSO: PBS at wavelengths of 270, 280, and 290 nm, respectively. Biopsy punches (6 mm) were placed in 2 ml centrifuge tubes. Samples were submerged in PBS and media was changed at 1 hour, 1 day, 7 days, 14 days, and 28 days. At these time points, media was transferred to another 2 ml centrifuge tube, diluted by 2 in DMSO to provide a 1:1 DMSO: PBS solution, and characterized using UV-vis to test release rates. Measurements were normalized to a blank consisting of 1:1 DMSO: PBS.

All cell viability testing was completed using Caco-2 cells (ATCC) incubated with physically and chemically incorporated samples (n=3). Cells were cultured in DMEM supplemented with 10% fetal bovine serum and 1% pen-strep. Cells were trypsinized and placed into a 24-well plate at a density of 5,000 cells per well. Transwell inserts containing 6-mm biopsy punch samples were then placed into testing wells. Cell viability was assessed at 24 hours using a resazurin assay (Alamar Blue) and measured fluorescence absorbances (excitation 530 nm and 590 emission) by a plate reader a (BioTek Synergy 2). Cell growth was assessed in CA and PCA gels over 7 days also using a resazurin assay. Ca-A hydrogels were not assessed for growth due to poor initial cytocompatibility. Cell viability was calculated as:

$$Cell Viability ( \% ) = \frac{{Abs}_{Sample} - {Abs}_{Blank}}{{Abs}_{Control} - {Abs}_{Blank}} \times 100$$

Where blanks contained media only, an the control contained cells with media. Cell growth was examined by comparing the relative absorbance values at 7 days to the initial absorbance after 24 hours. Relative absorbance was calculated by:

$$Relative absorbance = \frac{{Abs}_{Sample day 7} - {Abs}_{Blank}}{{Abs}_{sample day 1} - {Abs}_{Blank}}$$

Hydrogel samples (n=3) were cut into 6 mm diameter disks and UV sterilized for 20 minutes on each side. Antimicrobial properties were examined in Escherichia coli (E. coli). Bacterial was grown in 5 ml of lysogeny broth (LB) at 37° C. overnight. After 16 hours, 1 ml of the bacterial solution was diluted to 10 ml with LB. The solutions were then incubated until they reached an optical density of 0.6, which was confirmed by plate reader. Samples were placed into a 96-well plate and 100 μL of the bacterial solution was placed into each well. The plate was then incubated for 1 hour at 37° C. The solution in the well was diluted by 10⁷ in LB, and 10 μL of the bacterial solution from each well was drop-plated onto a LB-agar plate and incubated for 18 hours at 37° C. Images of the plates were taken at the completion of the incubation and examined for qualitative analysis of colony forming unit (CFU) density.

The effects of chemically and physically incorporated CA and PCA on biofilm formation was examined using Staphylococcus aureus (S. aureus). Overnight cultures of S. aureus were initially prepared similarly to E. coli preparation described in the antimicrobial testing. The bacteria solution was grown to an optical density of 0.4. Samples were placed into a 12-well plate with 2 ml of a 0.25% glucose+LB solution. Then 400 μL of the bacterial solution was added and incubated for 24 hours under static conditions. The samples were then prepared for either scanning electron microscopy (SEM) imaging of attached biofilm (n=3) or crystal violet (CV) staining of surrounding biofilm (n=3). For SEM imaging, the samples were washed 3 times with DI water and 3 times with 0.85% NaCl and then fixed using 2.5% glutaraldehyde for 1 hour. The samples were placed in a series of solutions with increasing acetone concentrations (15, 30, 50, 60, 75, and 100% in water) for 15 minutes each to dehydrate. The samples were air-dried overnight and imaged via SEM (Jeol NeoScope JCM-5000) at 1500 magnification and 15 kV.

For CV staining, samples were carefully removed from wells, and 400 μL of 0.1% CV was placed into the wells for 20 minutes. The well was then washed 3 times with DI water. The CV was redissolved by adding 30% acetic acid to each well. The solution was then read at an absorbance of 540 nm by a plate reader. Relative absorbance was calculated by:

$$Relative absorbance = \frac{{Abs}_{Sample}}{{Abs}_{Control}}$$

The control sample was a non-modified PVA film.

All hydrogel formulations containing CA and PCA were tested for shape memory. Ca-A was excluded due to poor cytocompatibility and antimicrobial properties. For chemically incorporated hydrogels, the samples were allowed to incubate in DI water overnight at 37° C. Films were then punched with a dog bone cutter, folded, and placed with the folded ends facing down into the single well of a 96-well plate. Placement into the well plate kept the samples from unfolding while drying. The samples were vacuum dried for 48 hours to fix the secondary shape. Physically incorporated dog bone-shaped hydrogels were prepared as mentioned above, but prior to drying they were folded and placed ends down into a 48-well plate. Larger well plates were used due to higher swelling and the more brittle nature of the hydrogels when swollen in DMSO during PA incorporation. After drying, samples were removed from wells and incubated for 24 hours at ambient humidity and room temperature. Shape fixity was assessed by tracking the change in the distance between the two folded ends and calculated as a percent length change using:

$$Length change ( \% ) = \frac{{Distance}_{24 hour} - {Distance}_{intital}}{{Distance}_{intital}} \times 100 \%$$

Measurements are presented as mean±standard deviation. Single factor Anova with Tukey's post hoc were used determine statistical significance. A p-value of <0.05 was taken as statistically significant.

| Type of PA | CA | | | PCA | | | Ca—A | | | Control |
|---|---|---|---|---|---|---|---|---|---|---|
| Amount | 1% | 2% | 5% | 1% | 2% | 5% | 1% | 2% | 5% | 0% |
| of PA | | | | | | | | | | |
| Gel | 98 ± | 94 ± | 100 ± | 92 ± | 94 ± | 83 ± | 85 ± | 89 ± | 77 ± | 99 ± |
| Fraction (%) | 2 | 9 | 3 | 18 | 2 | 1 | 2 | 10 | 4 | 4 |
| Swelling | 1.46 ± | 2.42 ± | 1.85 ± | 2.14 ± | 2.05 ± | 1.40 ± | 1.85 ± | 1.86 ± | 2.31 ± | 2.87 ± |
| Ratio | 0.02 | 0.67 | 0.01 | 0.11 | 0.01 | 0.11 | 0.02 | 0.68 | 0.01 | 0.03 |
| PA | 25 ± | 32 ± | 47 ± | 37 ± | 70 ± | 144 ± | 35 ± | 52 ± | 125 ± | N/A |
| Absorption | 14 | 1 | 14 | 16 | 17 | 26 | 16 | 27 | 3 | |
| (w/w %) | | | | | | | | | | |

Gel fractions for all chemically incorporated PA-PVA hydrogel formulations ranged from 77%-100%, as seen in Table 1 below. High gel fractions demonstrated successful crosslinking. In general, hydrogels containing Ca-A had the lowest gel fractions ranging between 77 and 85% compared to the highest gel fractions in CA-incorporated PVA hydrogels, which were between 94 and 100%. In general, swelling ratios were at 50% higher than the initial dry weight, and PA incorporation reduced swelling in comparison with the control. In CA hydrogels, swelling was highest in the 2% gels. In PCA hydrogels, swelling was similar in 1% and 2% gels and lowest in 5% gels. In Ca-A hydrogels, swelling was similar in 1% and 2% gels and highest in 5% gels.

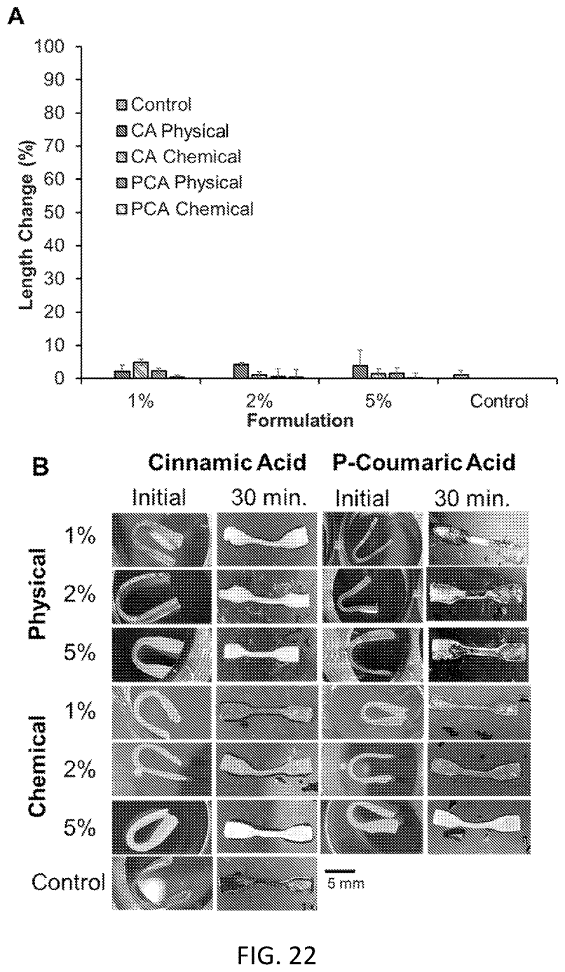

The FTIR spectra of PCA hydrogels are shown in FIG. 22, which provides a representation of general trends that were observed in all PA-containing hydrogels. A peak at ˜1700 cm⁻¹ is present in all formulations, representing the carbonyl of the urethane in the polymer backbone. In physically incorporated hydrogels (FIG. 22A), the aromatic stretching peak at ˜1600 cm⁻¹ increases with increased PA content. In both chemically and physically incorporated PA-PVA hydrogels (FIG. 22B), a shoulder peak is seen at −1620 cm⁻¹, which is the alkene bond in the phenolic ring (C═C).

Physically incorporated PA content: All physically incorporated PA-PVA hydrogels had an increase in PA absorption as the PA concentration increased from 1-5% (Table 1). CA showed the lowest incorporation percentage, with all CA formulations absorbing under 50% of the polymer weight in CA. PCA had the highest incorporation amount at all three PA solution concentrations. Incubation of samples in the 5% PCA and Ca-A solutions resulted in over 100% absorption of PAs relative to the polymer weight, with PCA having the greatest absorption at 144% of the polymer weight.

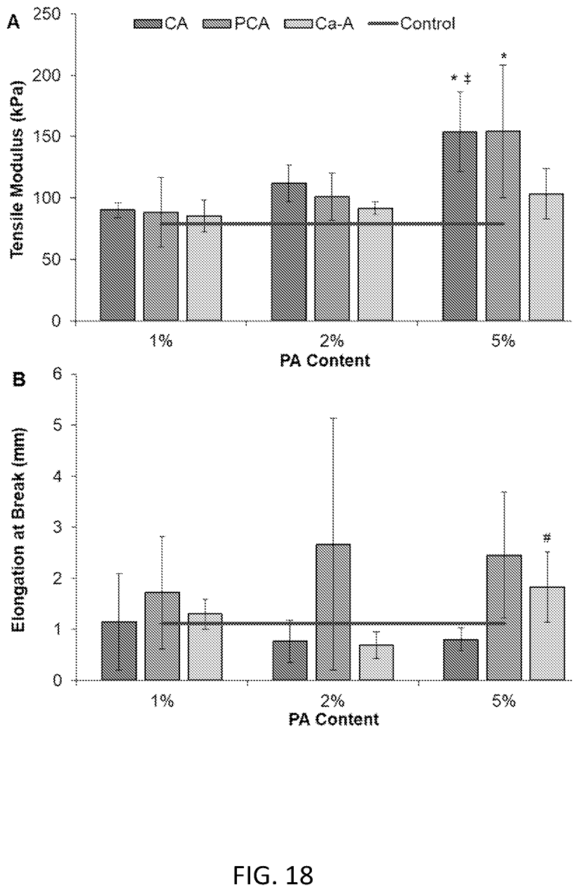

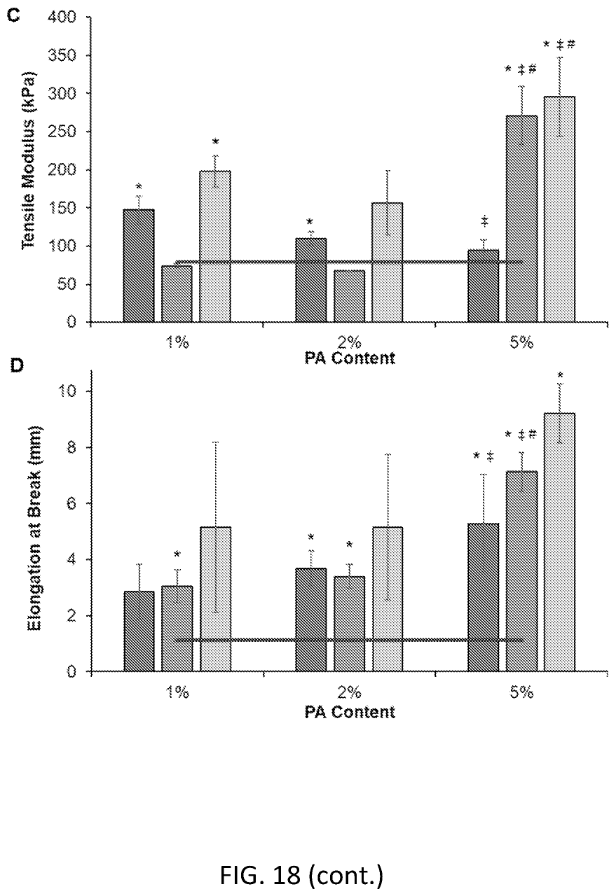

All physically incorporated PA-PVA hydrogels had a similar or higher modulus than the control of 79 kPa, FIG. 18A. In general, as the PA content was increased in physically incorporated PVA hydrogels, the modulus increased. Hydrogels with physically incorporated CA and PCA that contain 0-1 hydroxyl groups on the PA ring had larger increases in modulus as PA content increased. Higher concentrations of physically incorporated CA (2 and 5%) resulted in lower elongation at break compared with the control (no statistical difference), FIG. 18A. PCA gels had the highest elongation at break within the physically incorporated PA hydrogels, with an increase between 1 and 2% PCA and a plateau between 2 and 5% PCA. A high concentration of Ca-A (5%) also increased elongation at break compared with control.

As the chemically incorporated CA content increased, the modulus decreased to values that were comparable with that of the control, FIG. 18A. Chemically incorporated PCA hydrogels had constant modulus at 1 and 2% PCA, and a large increase in modulus (3.7-4×) was observed in 5% PCA gels. Chemically incorporated Ca-A hydrogels had the highest modulus at each concentration, with similar stiffnesses at 1 and 2% Ca-A content and increased stiffness at 5% Ca-A. Elongation at break was higher in chemically incorporated PA hydrogels. Generally, as the PA content and the number of pendant hydroxyls on chemically incorporated PAs increased, the elongation at break increased, FIG. 18D.

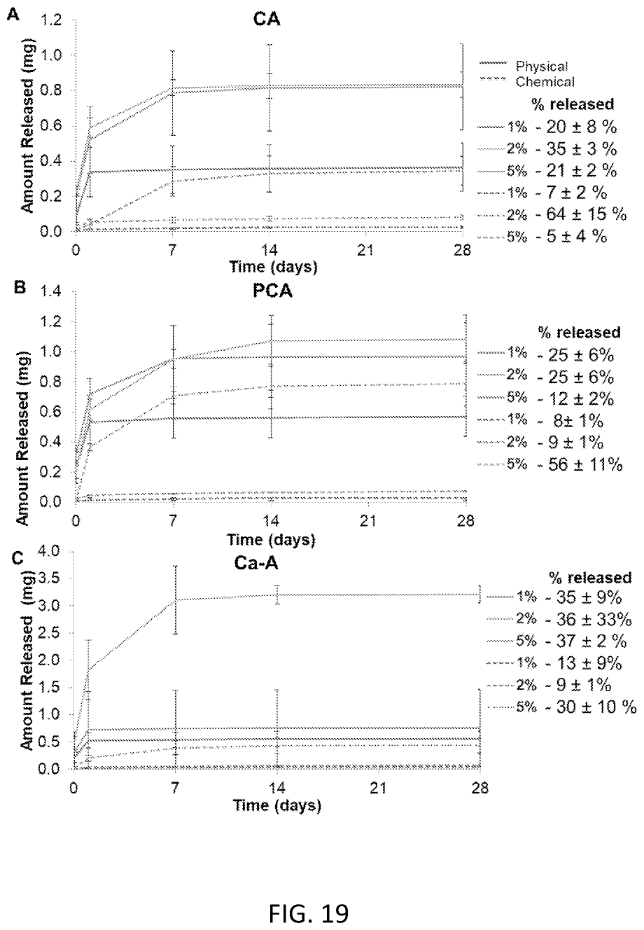

Hydrogels with physically incorporated PAs had an increased and sustained release compared to chemically incorporated PAs, FIG. 19, solid lines. All formulations showed a burst release of PA at 1 day. As PA content increased from 1 to 5%, PA release increased, and the point at which release plateaued was delayed from 1 day for 1% PA incorporation to 7-14 days for 5% PA incorporation. Thus, release was sustained for longer time frames in higher PA content gels. Physically incorporated Ca-A hydrogels showed the largest released PA content (35-37%) at all concentrations of PA loading.

In general, there were low levels of release in the majority of all chemically incorporated PA-PVA hydrogels, FIG. 19, dashed lines. CA containing hydrogels had the highest release in 5% CA content, and both PCA and Ca-A had higher, prolonged release in 2% formulations. Minimal PA release was observed after 14 days in the majority of physically and chemically incorporated formulations.

Chemically incorporated hydrogels generally had greater than 75% cell viability (based on ISO 10993-5 benchmark), except for 2 and 5% Ca-A formulations, FIG. 201A. For physically incorporated hydrogels, decreased cell viability was observed with increased CA and PCA content, with all physically incorporated CA hydrogels having >75% cell viability and only 1% PCA having viability >75% at 24 hours. Hydrogels with physically incorporated Ca-A all showed lower cell viability of 48, 61, and 58% for 1, 2, and 5% Ca-A, respectively. In terms of cell growth over 7 days (relative to cell numbers at 24 hours of incubation with each sample), all hydrogels promoted increases in cell numbers over time. However, physically incorporated PCA resulted in the slowest cell growth, FIG. 19B. Cell growth decreased with increasing concentration of CA, with 1% CA having the lowest impact on cell growth. Chemically-incorporated PAs only showed significant effects on cell growth relative to the control hydrogel at 5% modification.

Colony forming units of E. coli (1 hour incubation): None of the chemically incorporated PCA or Ca-A hydrogels showed qualitative effects on E. coli density in comparison with the PVA control after 1 hour of incubation, FIG. 20A. Chemically incorporated CA at 2 and 5% reduced CFU density considerably to a point where individual colonies could be seen (vs. ‘lawn’ of E. coli in controls, PCA, and Ca-A). Physically incorporated CA hydrogels showed no visible CFUs at all CA concentrations, showing improved antimicrobial properties to levels that were comparable to the silver-based wound dressing clinical control (Ag). Similarly, physically incorporated PCA hydrogels had little to no CFU formation. Low concentrations of physically incorporated Ca-A (1%) did not visibly affect CFU density, again resulting in ‘lawn’ formation. Higher concentrations of physically incorporated Ca-A reduced bacteria density to levels where individual colonies could be viewed, but still had relatively high CFU densities.

Staph. aureus biofilm formation (24-hour incubation): Visual assessment of biofilm by SEM showed decreased biofilm formation on physically incorporated PCA and CA hydrogel surfaces, FIG. 20C. Chemically incorporated PAs showed a limited effects on biofilm formation in comparison with controls. Crystal violet staining of biofilm in the surrounding well confirmed significant decreases in biofilm formation compared to the control in physically incorporated CA and PCA hydrogels, FIG. 20B. All physically incorporated CA hydrogels showed comparably low biofilm formation (0.40, 0.39, and 0.42 relative absorbance compared with PVA hydrogel control in 1, 2, and 5% CA, respectively). Hydrogels with 2% physically incorporated PCA had the best biofilm inhibition (0.33 relative absorbance), and 1 and 5% PCA showed 0.57 and 0.50 relative absorbance, respectively. In the chemically modified hydrogels, only 5% CA showed a significant reduction in biofilm formation compared with the control, and the relative reduction was lower than that of 5% physically incorporated CA.

Based on the promising antimicrobial and cytocompatibility properties of CA and PCA hydrogels, their shape fixity and recovery were characterized to evaluate shape memory properties. Some small shape changes were observed in samples that were fixed into a folded secondary shape after 24 hours of storage at ambient humidity and room temperature; however, all average changes were less than 5% of the original length (measured as distance between folded ends), indicating high shape fixity in these materials, FIG. 223A. All tested hydrogels show the ability to recover to their primary, unfolded state after 30 minutes in 37° C. water, FIG. 22B.

The gel fraction, FTIR, and PA absorption data all provide evidence of successful chemical (gel fraction and FTIR) and physical (FTIR and PA absorption) PA incorporation into PVA hydrogels. For chemically incorporated hydrogels, the gel fraction depends on both PA concentration and the number of phenolic hydroxyls. In general, gel fraction decreased in all formulations with PAs that have hydroxyl substituted rings (i.e., PCA and Ca-A). Specifically, CA, which does not have any phenolic hydroxyls, had the highest gel fractions at all concentrations, while Ca-A has the highest number of pendant hydroxyls (2) and the lowest gel fractions. This result may be attributed to reactions between phenolic hydroxyls on PCA and Ca-A and isocyanates, which would consume free isocyanate ends to limit crosslinking and lower gel fractions. The phenolic hydroxyls are less reactive than primary and secondary aliphatic alcohols. Thus, the reaction of the carboxylic acid end of PAs should be more efficient; however, it is possible that some undesired reactions occurred between the phenolic rings during incorporation. To improve gel fractions in future work, other methods of chemical incorporation may be employed, such as esterification of PA carboxylic acids with another monomer to provide hydroxyl or amine end groups with higher isocyanate reactivity.

Physical PA incorporation amount was dependent on the number of hydroxyls and the concentration of PAs in the swelling solution. At concentrations of 1%, the amount of PA absorbed into the material was between 24 and 35% of the polymer weight. This concentration of PAs is significant, as concentrations as low as 270 μM have shown to be effective for antimicrobial properties in prior work. Even at low concentrations, the loading shows dependence on the hydroxyl content of the PA, where PAs with increased hydroxyl content had higher loading efficiencies. This trend becomes more evident in hydrogels swollen in higher PA concentrations. At 5%, the PA content of PCA and Ca-A hydrogels was 3 times that of CA gels. The hydroxyls allow for an increased number of intermolecular interactions between PVA chains and the physically incorporated PAs. Namely, increasing the number of hydrogen bonding sites enables more efficient incorporation of PCA and Ca-A. Phenolic compounds were incorporated into physically crosslinked PVA hydrogels in prior work. These materials showed increased glass transition temperature, which was similarly attributed to the physical crosslinks created by hydroxyls on the phenolic compounds.

The addition of PAs both physically and chemically leads to changes in the material properties of the hydrogels. First, chemical incorporation affected hydrogel swelling. PA-modified PVA hydrogels had decreased swelling in all formulations compared to the control PVA hydrogel. CA incorporation into the hydrogels led to the largest decreases in swelling, which is attributed to its relatively hydrophobic ring with no hydroxyls that limits water interactions with the resulting hydrogels. A previous study showed that increased modification of chitosan with relatively hydrophobic tyramine led to decreased swelling. The modification of PVA with both CA and HDI increases the hydrophobic regions of the polymer by the addition of 6-carbon chains in HDI and a benzene ring in CA to produce a similar effect on swelling. PAs with hydrophilic hydroxyl-substituted benzene rings (PCA and Ca-A) showed reduced effects on swelling properties. Further examination on the effects of chemical modification concentrations is required in future work, as no clear trends were observed between swelling ratios and PA concentrations.

The effect of PA incorporation on mechanical properties was examined by tensile testing. Physical incorporation of PAs into hydrogels increased tensile modulus and affected ultimate elongation. General trends of increased modulus were observed with decreasing PA hydroxyl substitutions and/or increasing PA content. CA showed the largest effect on modulus at 1 and 2%, and PCA showed a similar modulus to that of CA at 5% incorporation. Larger amounts of chemically incorporated PAs (5%) increased modulus in all formulations. At lower concentrations, increased modulus was seen in CA and Ca-A, but not PCA. Hydrogels modified with CA substitute a pendent hydroxyl on the PVA chain with a benzene ring from CA. The incorporation of benzene rings into hydrogels has been shown to increase hydrogel moduli due to the bulky and restricted structure. In contrast, PCA seems to have a plasticizing effect at lower concentrations, separating chains to reduce intermolecular bonds, and decreasing the modulus in comparison to the control. This effect is similar to data previously published on poly(caprolactone)-based polymers with chemically incorporated PCA, which showed decreased modulus and increased elongation at break.

Improved elongation at break was seen in all chemically modified hydrogels and in physically modified hydrogels with PCA (all concentrations) and Ca-A (1 and 5%). Chemical modification of PVA-based hydrogels has previously shown to improve elongation at the break due to a decrease in the order of crystalline regions because of the disruption of highly regular hydroxyl repeats. Physical incorporation of CA decreased elongation, with increased effects as CA content increased, which matches the observed increases in stiffness with increased physically incorporated CA. Conversely, higher concentration of physically incorporated PCA and Ca-A generally increased elongation at break. Elasticity and modulus are important factors to consider for Crohn's fistula filling due to the peristaltic environment of the gastrointestinal tract. Typically, modulus (stiffness) is inversely related to elongation and these properties are difficult to tune independently. Incorporation of PAs provides a new tool to increase both properties simultaneously, which could be valuable in a range of materials applications.

In general, release rates of PAs were highest in physically incorporated hydrogels. All physically incorporated PAs showed a burst release over 24 hours, and release after 24 hours was dependent on PA concentration. PA release also correlated with loading efficiency, with higher release from hydrogels that had larger amounts of physically incorporated PA (and higher numbers of hydrogen bonds). Lower PA release was measured in chemically incorporated hydrogels due to the non-degradable nature of these materials. The amide linkage that forms between HDI and the PAs is non-degradable in a PBS-based solution. In general, the formulations with lower gel fractions showed higher PA release. Thus, by increasing gel fractions, the release of chemically incorporated PAs could be better predicted. Additionally, modifications in hydrogel washing could reduce unwanted PA release in future work. Due to the low solubility of PAs in water, removal of unreacted monomer may be higher in other solvents, such as DMSO.

Chemically incorporated PA hydrogels showed good cytocompatibility for all compositions, except for Ca-A at 2 and 5%. Cytocompatibility of physically incorporated hydrogels was reduced with increased PA concentration and increased PA ring hydroxyls, which correlates with the PA release data. CA showed the best cytocompatibility at 24 hours, with all CA concentrations having cytocompatibility above the 75% benchmark determined by ISO 10933-5. PCA was only cytocompatible at 1%, and PCA hydrogels generally showed the largest variability in cell testing. This result may be attributed to the ability of PCA to affect cell attachment. Ca-A materials all showed cell viability below the ISO standard, which is consistent with previous reports. Due to the cytotoxic nature of Ca-A, it was not examined for cell growth over 7 days. Both CA and PCA showed effects on cell growth over 7 days. Increasing PA concentration in the hydrogels slowed cellular growth. CA hydrogels showed superior growth in comparison to PCA. Both CA and PCA have been shown to inhibit mammalian cell growth at concentrations >600 uM. Based on this data, CA would likely be a better option for wound healing in Crohn's fistulas than PCA or Ca-A.

The PA release rate from the hydrogels also affected antimicrobial properties. All chemically incorporated PAs showed minimal effects on E. coli colony forming units, while physical incorporation of PAs decreased E. coli CFU formation. Interestingly, as the number of substituted hydroxyls increases, there is a decrease in overall antimicrobial ability, which corroborates with our previous results that showed moderate decreases in antimicrobial activity with increased PA ring pendant groups. In general, PAs represent an alternative option to potentially cytotoxic silver. PAs have also shown effectiveness against drug-resistant strains of S. aureus and S. epi., thereby proving a potential alternative to traditional antibiotics.

Due to the poor cytocompatibility and antimicrobial properties of Ca-A, only CA and PCA were examined for biofilm growth. Chemical incorporation did not show significant reductions in S. aureus biofilm formation, except in 5% CA gels. Both CA and PCA showed biofilm reduction at all PA concentrations in physically incorporated hydrogels. This result indicates that the antimicrobial properties of chemically immobilized PAs are reduced as compared with non-immobilized PAs. Future work could examine PA chemical modifications with varied release mechanisms (e.g., Schiff's base or other reversible click chemistries). These chemistries could be used to limit the burst release of PAs and the negative effects seen here on cell growth. Another option could be to modify PVA chains and incorporate them physically into a crosslinked PVA hydrogel to form a semi-interpenetrating network. By using higher molecular weight chemically unbound polymeric materials, the hydrogels could have more sustained PA release and fewer effects on cellular processes.

All the tested materials show water-responsive shape memory behavior. This property is potentially useful for implantation processes in Crohn's fistula. Previous work showed that by engineering salt leached foams, these hydrogels can be compressed and stored into a low-profile shape. Ideally, these compressed gels could be delivered to the fistula using a catheter, where they would then expand to recover their primary shape upon exposure to water in the fistula tract and heating to body temperature. This expansion process could fill the fistula tract to enable healing. The present invention shows full recovery (unfolding) of non-porous films within 30 minutes, which can be improved by converting these materials to the porous hydrogels used in our previous work. Additionally, by combining biodegradable elements, such as disulfides and natural polysaccharides, the hydrogel would degrade without needing secondary removal, and these degradation processes could be used to further tune PA release.

With the addition of PAs into a PVA-based hydrogel, it is possible to achieve antimicrobial properties, which may aid in improved fistula healing. Strong antimicrobial and antibiofilm properties were achieved by physically incorporating CA and PCA, and physical incorporation enabled tuning of swelling and mechanical properties. Chemical incorporation did not have a large impact on antimicrobial properties, but it provides a new tool for tuning material properties, such as swelling, modulus, and elongation at break. While the intended application of this material is fistula healing, these strategies could be employed in a range of potential applications, such as biomaterial coatings, chronic wound infection, and vaginal infections. Furthermore, these strategies could be translated to any biomaterial system with hydrogen bonding capabilities, such as poly(ethylene glycol) or polyurethanes.

What is claimed is:

1. A shape memory polymer scaffold for treatment of a fistula, comprising:
a shape memory polymer foam that can be triggered to expand from a compressed shape to an expanded shape in response to water and temperature, wherein the shape memory polymer foam comprises an amount of poly(vinyl alcohol), a biodegradable compound, and optionally a phenolic acid.

2. The shape memory polymer scaffold of claim 1, wherein the biodegradable compound is starch.

3. The shape memory polymer scaffold of claim 2, wherein the phenolic acid is vanillic acid.

4. The shape memory polymer scaffold of claim 3, wherein the vanillic acid is coupled to the amount of poly(vinyl alcohol) in place of a plurality of pendant hydroxyls of the poly(vinyl alcohol).

5. The shape memory polymer scaffold of claim 4, wherein the amount of poly(vinyl alcohol) with pendant vanillic acid is crosslinked with the starch to form a hydrogel network.

6. The shape memory polymer scaffold of claim 5, wherein the hydrogel network has a plurality of pores.

7. The shape memory polymer scaffold of claim 6, wherein the plurality of pores have a pore size of about 200 micrometers.

8. The shape memory polymer scaffold of claim 2, wherein the phenolic acid is selected from the group consisting of cinnamic acid, p-coumaric acid, ferulic acid, sinapic acid, caffeic acid, benzoic acid, 4-hydroxy benzoic acid, vanillic acid, syringic acid, protocatechuic acid, and gallic acid.

9. A method of treating a fistula, comprising the steps of:
providing a shape memory polymer foam in a compressed shape, wherein the shape memory polymer foam comprises an amount of poly(vinyl alcohol), a degradable compound, and optionally a phenolic acid;
positioning the shape memory polymer foam into a fistula; and
allowing the shape memory polymer foam to expand into an expanded shape from the compressed shape while positioned in the fistula.

10. The method of claim 9, wherein the degradable compound is starch.

11. The method of claim 10, wherein the phenolic acid is vanillic acid.

12. The method of claim 11, wherein the vanillic acid is coupled to the poly(vinyl alcohol) in place of a plurality of pendant hydroxyls of the poly(vinyl alcohol).

13. The method of claim 12, wherein the poly(vinyl alcohol) with pendant vanillic acid is crosslinked with the starch to form a hydrogel network.

14. The method of claim 13, wherein the hydrogel network has a plurality of pores.

15. The method of claim 14, wherein the plurality of pores have a pore size of about 200 micrometers.

16. The method of claim 9, wherein the phenolic acid is selected from the group consisting of cinnamic acid, p-coumaric acid, and caffeic acid.
